(19) United States
(12) Patent Application Publication
(10) Pub. No.: US 2023/0286964 A1
(43) Pub. Date: Sep. 14, 2023

(54) Liquid Bio-based Benzoxazine Resin Systems with Improved Processability and High Performance

(71) Applicants: Giuseppe Raffaello Palmese, Hainesport, NJ (US); Mengwen Yu, Philadelphia, PA (US); John Joseph La Scala, Wilmington, DE (US)

(72) Inventors: Giuseppe Raffaello Palmese, Hainesport, NJ (US); Mengwen Yu, Philadelphia, PA (US); John Joseph La Scala, Wilmington, DE (US)

(73) Assignees: DREXEL UNIVERISTY, Philadelphia, PA (US); The United States of America as Represented by the Secretary of the Army, Washington, DC (US)

(21) Appl. No.: 18/005,704

(22) PCT Filed: Jul. 23, 2021
(86) PCT No.: PCT/US2021/043035

§ 371 Date: Jan. 17, 2023

Related U.S. Application Data
(60) Provisional application No. 63/056,430, filed on Jul. 24, 2020.

(51) Int. Cl. C07D 413/06

(52) U.S. Cl. CPC C07D 413/06

(57) ABSTRACT

A series of bio-based benzoxazine monomers were developed. These benzoxazine monomers are liquid at room temperature with good processability due to their low viscosities at or near room temperature and their relatively low polymerization temperatures. Cured materials made from these bio-based benzoxazines have excellent thermal and mechanical properties.

### CROSS REFERENCE TO CORRESPONDING APPLICATIONS

This application claims the benefit of U.S. Provisional Application No. 63/056,430, filed on Jul. 24, 2020, the entire disclosure of which is hereby incorporated by reference as if set forth fully herein.

### STATEMENT OF GOVERNMENT INTEREST

This invention was made with government support under Contract Number W911NF-16-2-0225 awarded by the United States Army Research Laboratory. The Government has certain rights in this invention.

### BACKGROUND OF THE INVENTION

Benzoxazines are bicyclic heterocycles. As such, they consist of a benzene ring that is joined to a six-membered heterocyclic ring that contains one nitrogen and one oxygen atom.¹ The most common synthesis approach for benzoxazine monomers is the Mannich reaction using phenols, formaldehyde, and amines.²⁻⁴ Benzoxazine precursors can be thermally cured to produce highly crosslinked thermosets with near-zero shrinkage without the use of strong catalysts. Cured benzoxazines exhibit excellent electrical properties, fire resistance, and thermal properties, such as high char yield and high glass transition temperature (T_{g}). Based on these characteristics, polybenzoxazines are considered to have excellent potential in applications such as electronics, high temperature composites, and flame retardants.^{5,6}

A growing number of researchers have attempted to replace petroleum-based materials with bio-based materials to address environmental concerns and reduce use of scarce resources. Many bio-based compounds have been utilized to synthesize benzoxazine monomers that have comparable properties to that of petroleum-based polybenzoxazines; however, the methods employing these compounds have notable shortcomings that limit their use in industrial applications. One such shortcoming is that most benzoxazines are solid at room temperature, making them substantially more difficult to process into a neat resin or polymer blend. Additionally, cured benzoxazine materials exhibit brittleness due to their high crosslink density. In this invention, bio-based benzoxazine systems which have asymmetrical and mono-functional structures in order to make materials that are liquid at room temperature and have excellent thermal properties and high toughness.

It is understood that the functional groups of bio-based phenols impact processability. For example, steric hindrance from the number and position of functional groups on phenolic benzene rings affects the melting temperature and reaction onset temperature. To overcome this, Dubois' group proposed combining eugenol and phenol to synthesize partial, asymmetrical di-functional benzoxazines in order to increase the crosslink density of the network and lower the melting point. The eugenol derivative that produces good properties has both of the ortho- and para-positions occupied, whereas benzoxazines based on eugenol only include a functionality at the meta-position to connect with other precursors, which leads to a high onset temperature and low crosslink density. By increasing the ratio of phenol to eugenol, the Dubois group were able to increase the T_{g} to 220° C. from 120° C.⁷

Habibi's group adopted a similar idea but used vanillin- and cardanol-based benzoxazine to provide a wider processing window as compared to symmetrical, vanillin-based benzoxazines, Habibi's group was able to use the alkyl side chains from cardanol to lower the melting point to 101° C. from 218° C.⁸

Several research groups have tried to use furfurylamine to synthesize mono-functional benzoxazines with good properties. Chou and Liu synthesized phenol-furfurylamine- and BPA-furfurylamine-based benzoxazines. These groups found that furan rings could be disubstituted and then used to form a crosslinked network for phenol-furfurylamine-based mono-functional benzoxazines. They also compared thermal and mechanical properties of cured phenol-furfurylamine-based and BPA-furfurylamine-based benzoxazines and found that both had storage module above 3 GPa a T_{g} of and about 300° C. with a 240° C. post curing temperature.⁹

Wang and Liu also obtained a crosslinked network from bio-based polybenzoxazines using guaiacol and furfurylamine. The resulting cured materials also possessed good thermal stability. Varma's group used vanillin and furfurylamine to develop bio-based benzoxazines with high char yield. They studied the curing process and confirmed that the furan rings participated in the curing reaction and additionally found that the formyl groups from vanillin transferred to carboxylic groups and acted as a catalyst to accelerate polymerization with the elimination of carboxylic groups.¹⁰ In these previous works, all of the mono-furan-based benzoxazines that were employed were solid at room temperature.

Ishida and Jin used aniline and gastrodigenin, a natural phenolic compound, to synthesize benzoxazine and methacryloyl-functional benzoxazine. Ishida and Jin lowered the melting temperature of the benzoxazines below 50° C.¹¹

WO 2019/040407 (WO '407) relates to renewable benzoxazine monomers and polymers that utilize a variety of building blocks found in renewable plant biomass, which may be used as replacements for some petroleum-based polymers. The benzoxazine compounds of WO '407 may be prepared by reacting furfurylamine compounds, formaldehyde compounds, and phenols. The benzoxazine compounds may have a structure according to Formula (A).

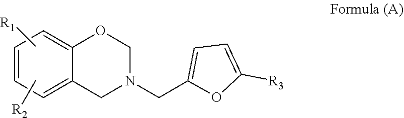

wherein R₁ and R₂ may independently be selected from hydrogen, a straight or branched alkyl, alkenyl, or alkoxy group each having 1-4 carbon atoms, and —C(═O)H; R₃ may be hydrogen or a group having the Formula (B):

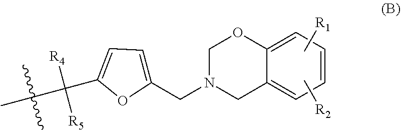

wherein R₁ and R₂ in Formula (B) may independently be selected from the same groups for R₁ and R₂ that are defined above,

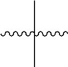

represents the bond to the ring carbon of the furan ring in Formula (A), and R₄ and R₅ in Formula (B) are each selected from hydrogen and an optionally substituted alkyl group having 1 to 20 carbon atoms, an optionally substituted alkenyl group having 2 to 20 carbon atoms, an optionally substituted cycloalkyl group having 3 to 12 carbon atoms, an optionally substituted aryl group having 6 to 16 carbon atoms, and an optionally substituted heterocyclic group having 3 to 16 carbon atoms; wherein the alkyl group, alkenyl group, cycloalkyl group, aryl group or heterocyclic group can be substituted with 1 to 5 substituents independently selected from a halogen, hydroxy, amino, nitro, cyano, carboxy, an alkyl group having 1 to 20 carbons, an aryl group having 6 to 16 carbon atoms, a heterocyclic group having 3 to 16 carbons, and an alkoxy group having 1 to 20 carbon atoms, and R₄ and R₅ in Formula (B) cannot both be hydrogen.

In the present disclosure, furan and gastrodigenin are used to develop lower melting point bio-based benzoxazines with excellent thermal properties. The thermal and mechanical properties of the furan-based polybenzoxazines were studied, as well as the effect of various furans on these properties.

### SUMMARY OF THE INVENTION

The present invention relates to benzoxazine compounds, polymers formed by ring opening polymerization of the benzoxazine compounds, and methods of preparing each of the foregoing.

The following sentences describe some embodiments of the invention.
1. In a first aspect, the disclosure relates to a benzoxazine compound selected from A)-C):
   A) a benzoxazine compound according to Formula (I):

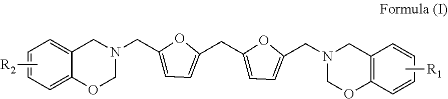

wherein R₁ and R₂ are each independently selected from hydrogen, a straight or branched alkyl group having 1 to 30 carbon atoms, a straight or branched alkenyl group having 5 to 30 carbon atoms, a straight or branched alkoxy group having 5 to 30 carbon atoms, or R_{A}OH, wherein R_{A} is a hydrocarbylene comprising 1 to 10 carbon atoms;
B) a benzoxazine compound according to Formula (II):

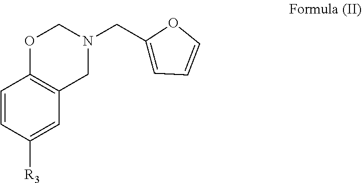

wherein R₃ is selected from a straight or branched alkyl group having 1 to 10 carbon atoms or from 5 to 10 carbon atoms, a straight or branched alkenyl group having 5 to 30 carbon atoms, or a straight or branched chain alkoxy group having 5 to 30 carbon atoms, or R_{B}OH, wherein R_{B} is a hydrocarbylene comprising 1 to 10 carbon atoms;
C) a benzoxazine compound according to Formula (III):

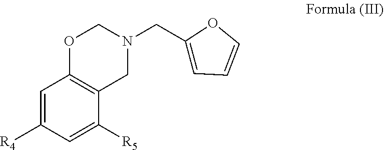

wherein R₄ and R₅ are each independently selected from a straight or branched alkyl group having 5 to 10 carbon atoms, a straight or branched alkenyl group having 5 to 30 carbon atoms, a straight or branched chain alkoxy group having 5 to 30 carbon atoms, or R_{C}OH, wherein R_{C} is a hydrocarbylene having 1 to 10 carbon atoms.
2. The benzoxazine compound of sentence 1, wherein the benzoxazine compound may be a compound of the Formula (I).
3. The benzoxazine compound of sentence 2, wherein R₁ may have from 7 to 25 carbon atoms, or from 10 to 20 carbon atoms.
4. The benzoxazine compound of any one of sentences 2 or 3, wherein R₁ may be an alkenyl group.
5. The benzoxazine compound of sentence 2, wherein each of R₁ and R₂ may be selected from hydrogen and R_{A}OH, and at least one of R₁ and R₂ is R_{A}OH; or R₁ and R₂ may each be R_{A}OH.
6. The benzoxazine compound of sentence 1, wherein the benzoxazine compound may be a compound according to the Formula (II).
7. The benzoxazine compound of sentence 6, wherein R₃ may be R_{B}OH.
8. The benzoxazine compound of sentence 1, wherein the benzoxazine compound may be a compound according to Formula (III).
9. The benzoxazine compound of sentence 8, wherein R₄ and R₅ may each be R_{C}OH.
10. In a second aspect, the present disclosure relates to a compound which is a reaction product prepared by the reaction of:
   i) a compound selected from:
   a) Formula (I) according to sentence 5,
   b) Formula (II) according to sentence 6, and
   c) Formula (III) according to sentence 9; and
   ii) a reagent selected from one of the following:
   a) a halo-containing epoxide which is preferably epichlorohydrin; and
   b) a radically polymerizable monomer.
11. The compound of sentence 10, wherein the compound may be a reaction product prepared from a compound of the Formula (I).
12. The compound of sentence 11, wherein the reagent may be the radically polymerizable monomer and may be selected from methacryloyl chloride, methacrylic anhydride, acryloyol chloride, acrylic anhydride, acrylic acid, methacrylic acid, and alkyl anhydrides comprising at least 2-20 carbon atoms and a reaction product according to Formula (IV) is formed:

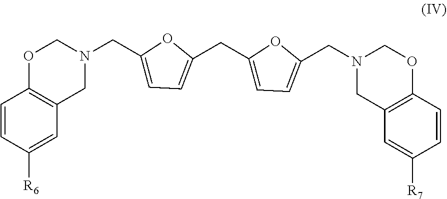

wherein R₆ and R₇ are each independently selected from hydrogen or a group having the Formula (X), wherein at least one of R₆ and R₇ is a group having the Formula (X):

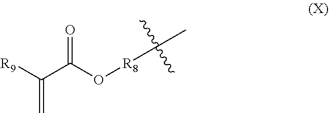

wherein R₈ in Formula (X) is a hydrocarbylene group comprising 1 to 10 carbon atoms, R₉ is selected from hydrogen, and a straight or branched alkyl group having 1 to 20 carbon atoms, and

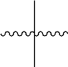

represents the bond attached to the ring carbon of the benzoxazine in Formula (IV).
13. The compound of sentence 12, wherein R₆ and R₇ may each be the group according to Formula (X).
14. The compound of sentence 11, wherein the reagent may be epichlorohydrin and a reaction product according to Formula (V) is formed:

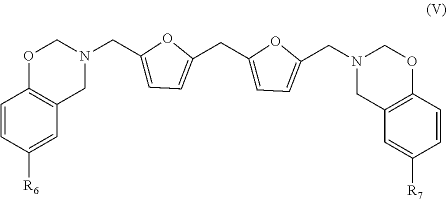

wherein R₆ and R₇ are each independently selected from hydrogen or a group having the Formula (XI), wherein at least one of R₆ and R₇ is a group having the Formula (XI):

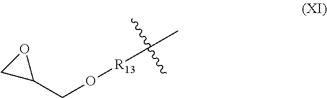

wherein R₁₃ is a hydrocarbylene group comprising 1 to 10 carbon atoms, and

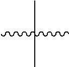

represents the bond attached to the ring carbon of the benzoxazine in Formula (V).
15. The compound of sentence 14, wherein R₆ and R₇ may each be the group according to Formula (XI).
16. The compound of sentence 10, wherein the compound may be a reaction product prepared from a compound of the Formula (II).
17. The compound of sentence 16, wherein the reagent may be the radically polymerizable monomer which may be selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms and a reaction product according to Formula (VI) is formed:

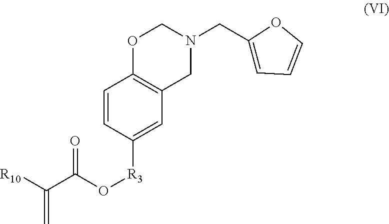

wherein R₃ is a hydrocarbylene group comprising 1 to 10 carbon atoms and R₁₀ is selected from hydrogen and a straight or branched alkyl group comprising 1 to 20 carbon atoms.
18. The compound of any one of sentences 16-17, wherein the reaction product may be (3-(furan-2-ylmethyl)-3,4-dihydro-2H-benzo[e][1,3]oxazin-6-yl)methyl methacrylate.
19. The compound of sentence 16, wherein the reagent may be epichlorohydrin and a reaction product according to Formula (VII) is formed:

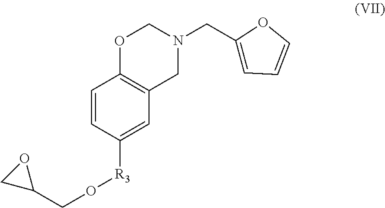

wherein R₃ is a hydrocarbylene group comprising 1 to 10 carbon atoms.
20. The compound of sentence 10, wherein the compound may be a reaction product prepared from a compound of the Formula (III).
21. The compound of sentence 20, wherein the reagent may be the radically polymerizable monomer which may be selected from methacryloyl chloride, methacrylic anhydride, acryloyol chloride, acrylic anhydride, acrylic acid, methacrylic acid, and alkyl anhydrides having 2 to 20 carbon atoms and a reaction product according to Formula (VIII) is formed:

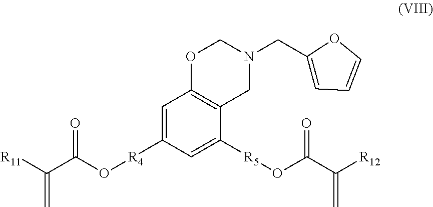

wherein R₄ and R₅ are each independently selected from a hydrocarbylene having 1 to 10 carbon atoms, and R₁₁ and R₁₂ are each independently selected from hydrogen, and straight or branched alkyl groups having 1 to 20 carbon atoms.
22. The compound of sentence 20, wherein the reagent may be epichlorohydrin and a reaction product according to Formula (IX) is formed:

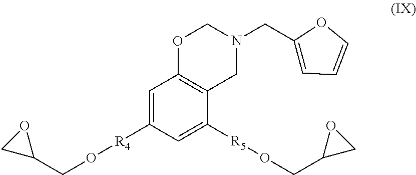

wherein R₄ and R₅ are each independently selected from a hydrocarbylene having 1 to 10 carbon atoms.
23. The benzoxazine compound of any one of sentences 1-22, wherein the benzoxazine compounds are liquids at room temperature.
24. In a third aspect, the present disclosure relates to a method of forming the benzoxazine compound of the Formula (I) of sentence 5, comprising a step of reacting a difurfuryldiamine, a formaldehyde compound, a phenolic compound, and a cardanol compound.
25. The method of sentence 24, wherein a molar ratio of the phenolic compound to the cardanol compound in the reacting step may be from 90:10 to 5:95, or from 80:20 to 40:60, or from 80:20 to 50:50.
26. The method of any one of sentences 24-25, wherein the phenolic compound may be selected from phenol, guaiacol, syringol, cardonal, and capsaicin, more preferably, phenol, cardanol, and guaiacol.
27. The method of any one of sentences 24-26, wherein the method may form a benzoxazine compound that is liquid at room temperature.
28. The method of any one of sentences 24-27, may further comprise a step of heating the reaction mixture at a temperature between 50° C. to 110° C., or from 60° C. to 100° C., or from 65° C. to 95° C.
29. The method of sentence 28, wherein the heating step may be carried out for 1 hour to 48 hours, or from 5 hours to 36 hours, or from 10 hours to 24 hours.
30. In a fourth aspect, the present disclosure relates to a method of forming the benzoxazine compound of the Formula (II) of sentence 7, comprising reacting a furfuryl amine, a formaldehyde compound, and a phenolic compound to form a first reaction product.
31. The method of sentence 30, wherein the reacting step may be carried out with a molar ratio of furfuryl amine to formaldehyde compound of from 1:4 to 1:1, or 1:2.
32. The method of any one of sentences 30-31, wherein the reacting step may be carried out with a molar ratio of furfuryl amine to phenolic compound of from 1:6 to 1:1, or from 1:4 to 1:2, or 1:3.
33. The method of any one of sentences 30-32, wherein the reacting step may be carried out at a temperature between 50° C. to 120° C., or from 60° C. to 100° C., or from 70° C. to 90° C.
34. The method of any one of sentences 30-33, wherein the reacting step may be carried out for 1 hour to 48 hours, or from 5 hours to 36 hours, or from 10 hours to 24 hours
35. The method of any one of sentences 30-34, wherein the phenolic compound may be substituted at the para-position.
36. The method of any one of sentences 30-35, wherein the phenolic compound may be selected from 4-hydroxybenzyl alcohol, 2-(4-hydroxyphenyl) ethanol, and p-cresol.
37. The method of any one of sentences 30-36, wherein the formaldehyde compound may be selected from formaldehyde and paraformaldehyde.
38. In a fifth aspect, the present disclosure relates to a method of forming the benzoxazine compound of the Formula (III) of sentence 9, comprising a step of reacting a furfuryl amine (50 g, 51.5 mmol), a formaldehyde compound (30.92 g, 103 mmol to 46.38 g, 154.5 mmol), and a 3,5 (hydroxyalkyl)phenolic compound (79.36 g, 51.5 mmol to 87.30 g, 56.6 mmol) to form a first reaction product, wherein the 3,5 (hydroxyalkyl) phenolic compound is preferably 3,5(hydroxymethyl) phenol.
39. The method of sentence 38, wherein the reacting step may be carried out with a molar ratio of furfuryl amine to formaldehyde to the 3,5 (hydroxyalkyl)phenolic compound of from about 1:1:1 to about 1:3:1, or from about 1:1:1 to 1:2:1.
40. In a sixth aspect, the present disclosure relates to a method of forming a benzoxazine reaction product comprising steps of:
a) forming a first reaction product as described in any one of sentences 24, 30, and 38; and
b) reacting a first reaction product with a reagent selected from one of the following:
i) a halo-containing epoxide which is preferably epichlorohydrin; and
ii) a radically polymerizable monomer;
in the presence of a base catalyst.
41. The method of sentence 40, wherein the reagent may be the radically polymerizable monomer and is selected from methacryloyl chloride, methacrylic anhydride, acryloyol chloride, acrylic anhydride, acrylic acid, methacrylic acid, and alkyl anhydrides comprising from 2 to 20 carbon atoms.
42. The method of sentence 40, wherein the reagent may be epichlorhydrin.
43. The method of any one of sentences 40-42, wherein the base catalyst may be selected from dimethylaminopyridine, trimethylamine, 1,8-diazabicyclo[5.4.0]undec-7-ene, 1-methylimidazole and 2-methylimidazole, and triethylamine.
44. The method of any one of sentences 40-43, wherein the method may form a benzoxazine compound that is liquid at room temperature.
45. The method of any one of sentences 40-44, wherein the reacting step b) may be carried out with a molar ratio of the first reaction product to the reagent of from about 1:4 to 2:1, or from about 1:2 to 1:1.
46. The method of any one of sentences 40-45, wherein reacting step b) may be carried out with a molar ratio of the first reaction product to the reagent to the base catalyst of from about 5:6:1 to 1:1:1, or from 5:5.5:1.5 to 1:1:1.
47. The method of any one of sentences 40-46, wherein the reacting step b) may be carried out in the presence of a solvent.
48. The method of sentence 47, wherein the solvent may be selected from dichloromethane, tetrahydrofuran, chloroform, and dimethylformamide, preferably, dichloromethane.
49. The method of sentence 41, wherein the first reaction product may be formed by the method of sentence 24, the reagent may be the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms; and a reaction product according to Formula (IV) is formed:

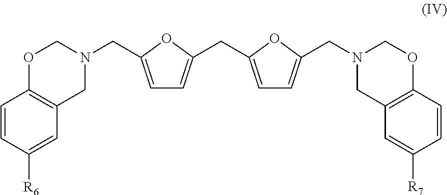

wherein R₆ and R₇ are each independently selected from hydrogen or a group having the Formula (X), wherein at least one of R₆ and R₇ is a group having the Formula (X):

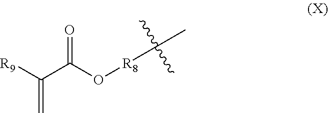

wherein R₈ in formula (X) is a hydrocarbylene group comprising 1 to 10 carbon atoms, R₉ is selected from hydrogen, and a straight or branched alkyl group having 1 to 20 carbon atoms, and

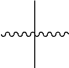

represents the bond attached to the ring carbon of the benzoxazine in Formula (IV).
50. The method of sentence 41, wherein the first reaction product may be formed by the method of sentence 24, the reagent is epichlorohydrin and a reaction product according to Formula (V) is formed:

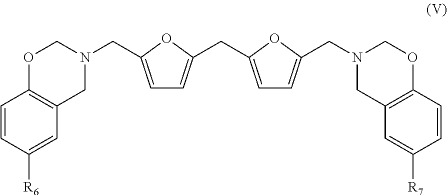

wherein R₆ and R₇ are each independently selected from hydrogen or a group having the Formula (XI), wherein at least one of R₆ and R₇ is a group having the Formula (XI):

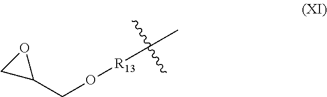

wherein R₁₃ is a hydrocarbylene group comprising 1 to 10 carbon atoms, and

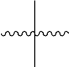

represents the bond attached to the ring carbon of the benzoxazine in Formula (V).
51. The method of sentence 41, wherein the first reaction product may be formed by the method of sentence 30, the reagent is the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms and a reaction product according to Formula (VI) is formed:

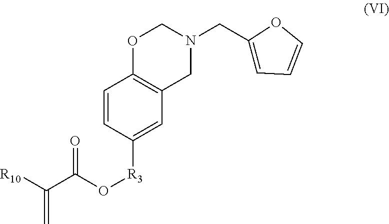

wherein R₃ is a hydrocarbylene group comprising 1 to 10 carbon atoms and R₁₀ is selected from hydrogen and a straight or branched alkyl group comprising 1 to 20 carbon atoms.
52. The method of sentence 42, wherein the first reaction product may be formed by the method of sentence 30, the reagent is epichlorohydrin and a reaction product according to Formula (VII) is formed:

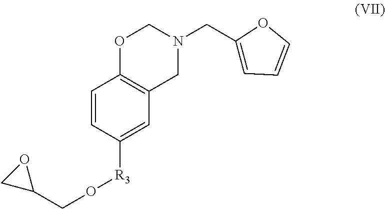

wherein R₃ is a hydrocarbylene having 1 to 10 carbon atoms.
53. The method of sentence 41, wherein the first reaction product may be formed by the method of sentence 38, the reagent is the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms and a product according to Formula (VIII) is formed:

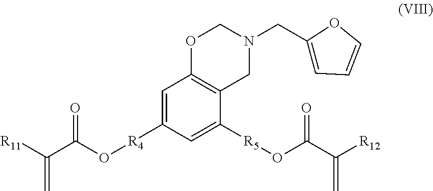

wherein R₄ and R₅ are each independently selected from a hydrocarbylene having 1 to 10 carbon atoms, and R₁₁ and R₁₂ are each independently selected from hydrogen, and straight or branched alkyl group having 1 to 20 carbon atoms.
54. The method of sentence 43, wherein the first reaction product may be formed by the method of sentence 38, the reagent is epichlorohydrin and a reaction product according to Formula (IX) is formed:

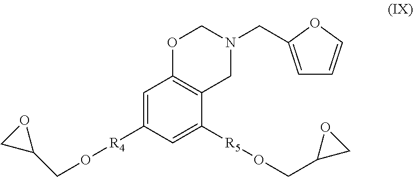

wherein R₄ and R₅ are each independently selected from a hydrocarbylene having 1 to 10 carbon atoms.
55. In a seventh aspect, the present disclosure relates to a polymer formed by ring opening polymerization of at least one benzoxazine compound of any one of the Formulas (I)-(III) of sentence 1, Formula (IV) of sentence 12, Formula (V) of sentence 14, Formula (VI) of sentence 17, Formula (VII) of sentence 19, Formula (VIII) of sentence 21, or Formula (IX) of sentence 22.
56. In an eighth aspect, the present disclosure relates to a benzoxazine compound that is a liquid at room temperature, according to Formulae (II)-(III):

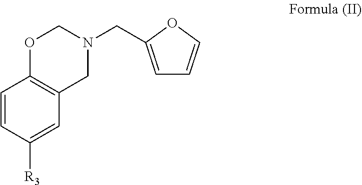

wherein R₃ is selected from a straight or branched alkyl group having 1 to 10 carbon atoms, a straight or branched alkenyl group having 1 to 30 carbon atoms, or a straight or branched chain alkoxy group having 1 to 30 carbon atoms, or R_{B}OH, wherein R_{B} is a hydrocarbylene comprising 1 to 10 carbon atoms; and
a benzoxazine compound according to Formula (III):

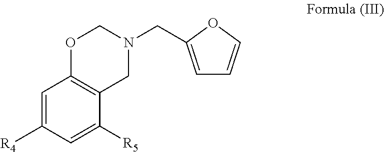

wherein R₄ and R₅ are each independently selected from a straight or branched alkyl group having 1 to 10 carbon atoms, a straight or branched alkenyl group having 1 to 30 carbon atoms, a straight or branched chain alkoxy group having 5 to 30 carbon atoms, or R_{C}OH, wherein R_{C} is a hydrocarbylene having 1 to 10 carbon atoms.
57. The benzoxazine compound of sentence 6, wherein R₃ is a straight or branched alkyl group having 5 to 10 carbon atoms.
58. In a ninth aspect, the present disclosure relates to a method of forming the benzoxazine compound of the Formula (II) of sentence 6, comprising reacting a furfuryl amine, a formaldehyde compound, and a phenolic compound to form a first reaction product.
59. The method of sentence 58, wherein the phenolic compound is substituted at the para-position or the meta-position.
60. The method of any one of sentences 58-59, wherein the phenolic compound is selected from m-cresol, p-cresol, and 4-ethylphenol.
61. In a tenth aspect, the present disclosure relates to a benzoxazine compound selected from the group consisting of compounds of the formulae A)-F):

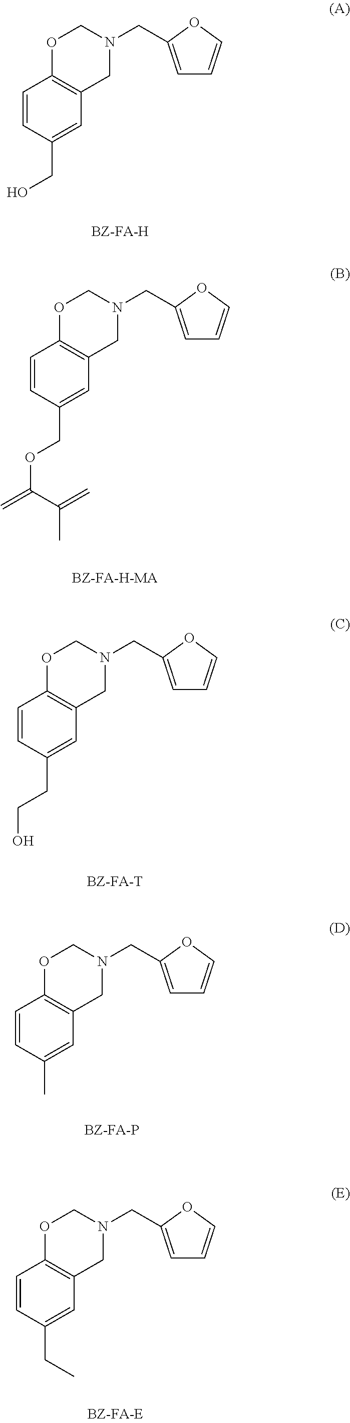 [SMILES: OCc1ccc2c(c1)CN(Cc1ccco1)CO2 ; C=C(C)C(=C)OCc1ccc2c(c1)CN(Cc1ccco1)CO2 ; OCCc1ccc2c(c1)CN(Cc1ccco1)CO2 ; Cc1ccc2c(c1)CN(Cc1ccco1)CO2 ; CCc1ccc2c(c1)CN(Cc1ccco1)CO2]

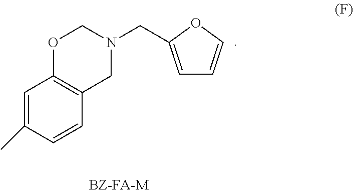 [SMILES: Cc1ccc2c(c1)OCN(Cc1ccco1)C2]

62. In an eleventh aspect, the present disclosure relates to a copolymer made by copolymerizing a mixture comprising:
a first benzoxazine compound according to sentence 7, wherein R₃ is R_{B}OH, wherein R_{B} is a hydrocarbylene comprising 1 to 3 carbon atoms; and
a second benzoxazine compound according to sentence 6, wherein R₃ is an alkyl group having 1 to 3 carbon atoms.
63. The copolymer of sentence 62, wherein a molar ratio of the first benzoxazine compound to the second benzoxazine compound in the mixture is from 90:10 to 5:95, or from 80:20 to 40:60, or from 80:20 to 50:50.
64. The copolymer of any one of sentences 62-63, wherein the first benzoxazine compound is:

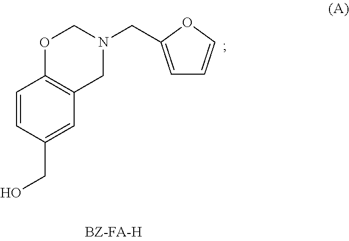 [SMILES: OCc1ccc2c(c1)CN(Cc1ccco1)CO2]

and the second benzoxazine compound is:

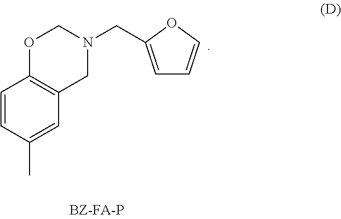 [SMILES: Cc1ccc2c(c1)CN(Cc1ccco1)CO2]

65. A method of preparing the copolymer of sentence 64, comprising
forming the first benzoxazine compound of Formula (A), comprising reacting a furfuryl amine, a formaldehyde compound, and 4-ethylphenol;
forming the second benzoxazine compound of Formula (D), comprising reacting a furfuryl amine, a formaldehyde compound, and p-cresol; and
copolymerizing the first benzoxazine compound and the second benzoxazine compound.

### BRIEF DESCRIPTION OF THE DRAWINGS

FIG. 1 shows a reaction mechanism for making (DFDA)-based asymmetrical benzoxazine blends.

FIG. 2 shows a ¹H-NMR spectrum of difurfuryldiamine based benzoxazines (BZ-DFDA)-Phenol/Cardanol blends.

FIG. 3 shows a reaction mechanism for making methacryloyl-functionalized benzoxazine.

FIG. 4A shows a ¹H-NMR spectra of (3-(furan-2-ylmethyl)-3,4-dihydro-2H-benzo[e][1,3]oxazin-6-yl)methanol (BZ-FA-H).

FIG. 4B shows a ¹H-NMR spectra of (3-(furan-2-ylmethyl)-3,4-dihydro-2H-benzo[e][1,3]oxazin-6-yl)methyl methacrylate (BZ-FA-H-MA).

FIG. 5 shows differential scanning calorimetry (DSC) curves for BZ-DFDA-Phenol/Cardanol.

FIG. 6A shows dynamic mechanical analysis (DMA) data of storage modulus for PolyBZ-DFDA-Phenol/Cardanol blends.

FIG. 6B shows DMA data of loss modulus for PolyBZ-DFDA-Phenol/Cardanol blends.

FIG. 6C shows DMA data of Tan Delta for PolyBZ-DFDA-Phenol/Cardanol blends.

FIG. 7 shows thermogravimetric analysis (TGA) thermograms for PolyBZ-DFDA-Phenol/Cardanol blends.

FIG. 8A shows Fourier-transform infrared spectroscopy (FTIR) spectra for BZ-FA-H and BZ-FA-H-MA.

FIG. 8B shows FTIR spectra for BZ-FA-H and BZ-FA-H-MA.

FIG. 9 shows DSC curves for BZ-FA-H and BZ-FA-H-MA.

FIG. 10A shows the viscosity of BZ-FA-H at various shear rates and temperatures.

FIG. 10B shows the viscosities of BZ-FA-H and BZ-FA-H-MA versus temperature.

FIG. 11A shows the DMA data of PolyBZ-FA-H.

FIG. 11B shows the DMA data of PolyBZ-FA-H-MA.

FIG. 12 shows TGA thermograms of PolyBZ-FA-H and PolyBZ-FA-H-MA.

FIG. 13 shows the strain versus stress curve for PolyBZ-FA-H

FIG. 14A shows DMA curves of the storage modulus of PolyBZ-FA-H for different post curing temperatures.

FIG. 14B shows DMA curves of the loss modulus of PolyBZ-FA-H for different post curing temperatures.

FIG. 15 shows the structures of the mono-furan based benzoxazines.

FIG. 16 shows the viscosity of various mono-furan based benzoxazines versus temperature.

FIG. 17 shows DMA data for polyBZ-FA-M.

FIG. 18 shows the viscosity of various BA-FA-H/BA-Fa/P blends versus temperature.

FIG. 19 shows DMA data for various PolyBZ-FA-H/BZ-FA-P copolymers.

FIG. 20 shows a ¹H-NMR spectra for BZ-DFDA-H.

FIG. 21 shows DMA data for PolyBZ-DFDA-H.

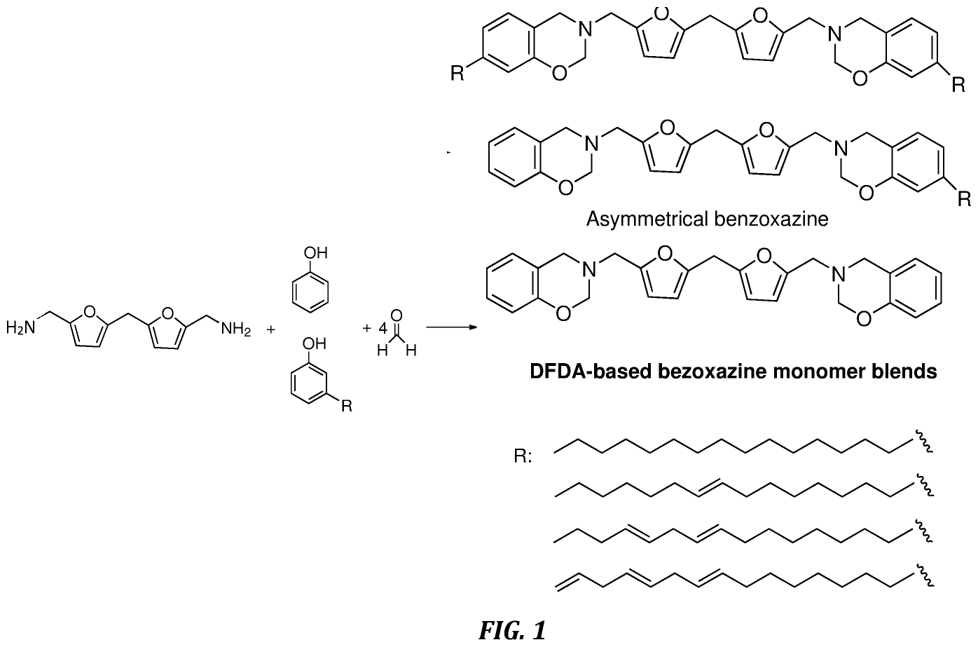

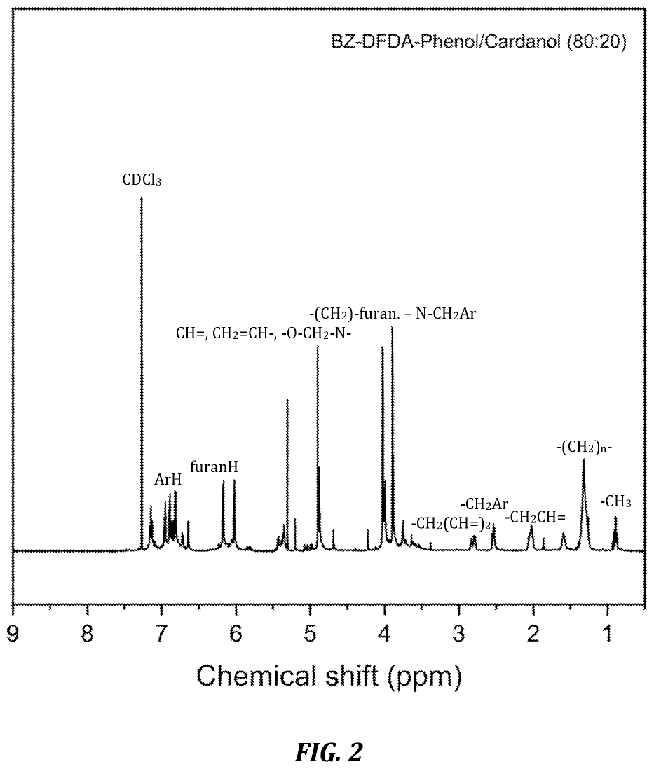

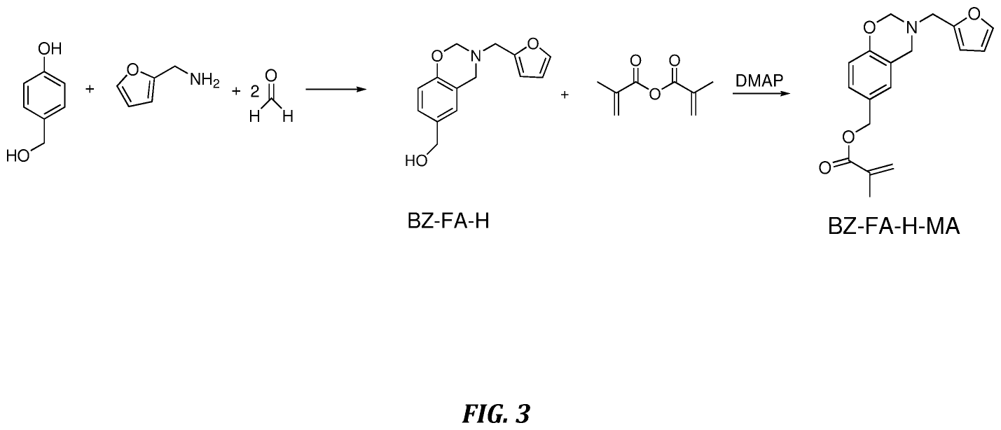

### DETAILED DESCRIPTION OF EMBODIMENTS

The present disclosure relates to benzoxazine compounds, polymers formed by ring opening polymerization of the benzoxazine compounds, and methods of preparing each of the foregoing.

### Benzoxazines

The benzoxazines of the present disclosure may be selected from any of the compounds A)-C) shown below:

Benzoxazine A) may be a compound according to Formula (I):

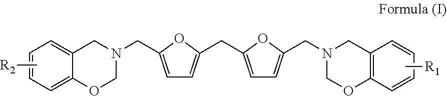

wherein R₁ and R₂ are each independently selected from hydrogen, a straight or branched alkyl group having 1 to 30 carbon atoms, a straight or branched alkenyl group having 5 to 30 carbon atoms, a straight or branched alkoxy group having 5 to 30 carbon atoms, or R_{A}OH, wherein R_{A} is a hydrocarbylene comprising 1 to 10 carbon atoms.

In some embodiments, R₁ may have from 7 to 25 carbon atoms, or from about 10 to 20 carbon atoms, or from about 12 to 18 carbon atoms.

In some embodiments, R₁ may be an alkenyl group having from 7 to 25 carbon atoms, or from about 10 to 20 carbon atoms, or from about 12 to 18 carbon atoms. In some embodiments, R₁ may be an alkyl group having from 7 to 25 carbon atoms, or from about 10 to 20 carbon atoms, or from about 12 to 18 carbon atoms.

In some embodiments, R₁ and R₂ may each independently be selected from hydrogen and R_{A}OH, and at least one of R₁ and R₂ is R_{A}OH; or R₁ and R₂ may each be R_{A}OH. When R₁ and R₂ are each R_{A}OH, each R_{A} may be independently selected from a hydrocarbylene comprising 1 to 10 carbon atoms.

Benzoxazine B) may be a compound according to Formula (II):

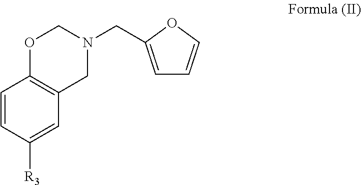

wherein R₃ is selected from a straight or branched alkyl group having 5 to 10 carbon atoms, a straight or branched alkenyl group having 5 to 30 carbon atoms, or a straight or branched chain alkoxy group having 5 to 30 carbon atoms, or R_{B}OH, wherein R_{B} is a hydrocarbylene comprising 1 to 10 carbon atoms.

In some embodiments, R₃ may be R_{B}OH. In some embodiments, R₃ is an alkyl group having from 6 to 8 carbon atoms, or an alkenyl group having from 5 to 20 carbon atoms, or from about 5 to 15 carbon atoms.

Benzoxazine C) may be a compound according to Formula (III):

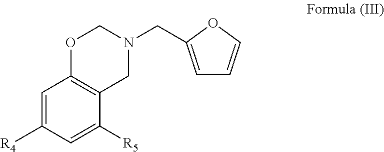

wherein R₄ and R₅ are each independently selected from a straight or branched alkyl group having 5 to 10 carbon atoms, a straight or branched alkenyl group having 5 to 30 carbon atoms, a straight or branched chain alkoxy group having 5 to 30 carbon atoms, or R_{C}OH, wherein R_{C} is a hydrocarbylene having 1 to 10 carbon atoms.

In some embodiments, R₄ and R₅ may each be R_{C}OH, wherein each R_{C} is independently selected from a hydrocarbylene having 1 to 10 carbon atoms.

The benzoxazine compounds according to any one of Formulas (I)-(III) may be a liquid at room temperature, wherein room temperature may be a temperature of about 20° C. to 25° C.

### Method of Preparing the Benzoxazines

Method of Preparing a First Reaction Product, Benzoxazine Compound (A) According to Formula (I), Wherein at Least One of R₁ and R₂ Comprises the Group R_{A}OH.

This method comprises a step of reacting a difurfuryldiamine, a formaldehyde compound, a phenolic compound, and a cardanol compound. Preferably, a molar ratio of the phenolic compound to the cardanol compound in the reacting step may be from 90:10 to 5:95, or from 80:20 to 40:60, or from 80:20 to 50:50.

Suitable examples of the phenolic compound are phenol guaiacol, syringol, cardanol, and capsaicin. Preferably, the phenolic compound is selected from phenol, cardanol, and guaiacol.

The method may include an additional step of heating the reaction mixture to a temperature between 50° C. to 110° C., or from 60° C. to 100° C., or from 65° C. to 95° C. The heating step may be carried out for a period of about 1 hour to 48 hours, or from about 5 hours to 36 hours, or from 10 hours to about 24 hours.

Method of Preparing a Second Reaction Product, Using the First Reaction Product, Benzoxazine Compound (A) According to Formula (I), Wherein at Least One of R₁ and R₂ Comprises the Group R_{A}OH.

This method of preparing the second reaction product comprises a first step of preparing the first reaction product, benzoxazine compound (A) according to Formula (I), as set forth above. The first reaction product is then reacted with a halo-containing epoxide reagent, preferably epichlorohydrin, and a radically polymerizable monomer reagent, in the presence of a base catalyst. Preferably, this reacting step employs a molar ratio of the first reaction product to the reagent of from about 1:4 to 2:1, or from about 1:2 to 1:1. In some embodiments, this reacting step of the method employs a molar ratio of the first reaction product to the reagent to the base catalyst of from about 5:6:1 to 1; 1:1, or from 5:5.5:1.5 to 1:1:1.

Exemplary radically polymerizable monomer may be selected from methacryloyl chloride, methacrylic anhydride, acryloyol chloride, acrylic anhydride, acrylic acid, methacrylic acid, and alkyl anhydrides comprising from 2 to 20 carbon atoms.

The base catalyst may be selected from dimethylaminopyridine, trimethylamine, 1,8-diazabicyclo[5.4.0]undec-7-ene, 1-methylimidazole and 2-methylimidazole, and triethylamine.

In some embodiments, the reacting step is carried out in the presence of a solvent. Suitable examples of the solvent are dichloromethane, tetrahydrofuran, chloroform, and dimethylformamide, preferably, dichloromethane.

When the reagent is a radically polymerizable monomer selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms, a second reaction product according to Formula (IV) is formed:

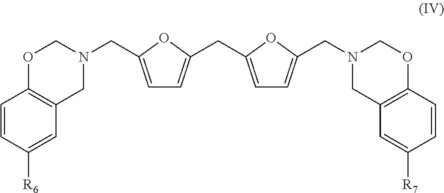

wherein R₆ and R₇ are each independently selected from hydrogen or a group having the Formula (X), and wherein at least one of R₆ and R₇ is a group having the Formula (X):

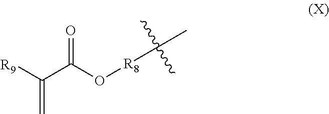

wherein R₈ in formula (X) is a hydrocarbylene group comprising 1 to 10 carbon atoms, R₉ is selected from hydrogen, and a straight or branched alkyl group having 1 to 20 carbon atoms, and

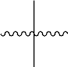

represents the bond attached to the ring carbon of the benzoxazine of the Formula (IV).

When the reagent is an epichlorohydrin a second reaction product according to Formula (V) is formed:

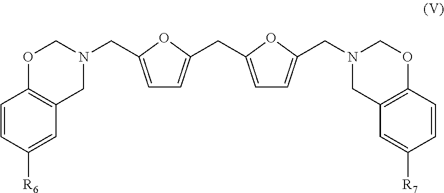

wherein R₆ and R₇ are each independently selected from hydrogen or a group having the Formula (XI), and wherein at least one of R₆ and R₇ is a group having the Formula (XI):

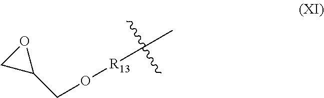

wherein R₁₃ is a hydrocarbylene group comprising 1 to 10 carbon atoms, and

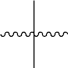

represents the bond attached to the ring carbon of the benzoxazine in Formula (V).

Method of Preparing a First Reaction Product, Benzoxazine Compound (B) According to Formula (II), Wherein R₃ is R_{B}OH

This method comprises a step of reacting a furfuryl amine, a formaldehyde compound, and a phenolic compound to form a first reaction product. Preferably, a molar ratio of the furfuryl amine to formaldehyde compound in this reacting step is from about 1:4 to 1:1, or about 1:2. Preferably, a molar ratio of furfuryl amine to phenolic compound in this reacting step is from 1:6 to 1:1, or from 1:4 to 1:2, or about 1:3.

The method may include an additional step of heating the reaction mixture at a temperature between 50° C. to 120° C., or from 60° C. to 100° C., or from 70° C. to 90° C. This heating step may be carried out for about 1 hour to 48 hours, or from about 5 hours to 36 hours, or from about 10 hours to 24 hours.

Preferably, the phenolic compounds may be substituted at the para-position. Suitable examples of such substituted phenolic compounds may be selected from 4-hydroxybenzyl alcohol, 2-(4-hydroxyphenol) ethanol, and p-cresol.

Suitable examples of the formaldehyde compound are formaldehyde and paraformaldehyde.

Method of Preparing a Second Reaction Product, Using the First Reaction Product, Benzoxazine Compound (B) According to Formula (II), Wherein R₃ is R_{B}OH.

This method of preparing the second reaction product comprises first preparing the first reaction product, benzoxazine compound (B) according to Formula (II), as set forth above. The first reaction product is then reacted with either a halo-containing epoxide reagent, preferably epichlorohydrin, or a radically polymerizable monomer reagent, in the presence of a base catalyst.

Suitable examples of the radically polymerizable monomer may be selected from methacryloyl chloride, methacrylic anhydride, acryloyol chloride, acrylic anhydride, acrylic acid, methacrylic acid, and alkyl anhydrides comprising from 2 to 20 carbon atoms.

Suitable examples of the base catalyst may be selected from dimethylaminopyridine, trimethylamine, 1,8-diazabicyclo[5.4.0]undec-7-ene, 1-methylimidazole and 2-methylimidazole, and triethylamine.

When the reagent is the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms a second reaction product according to Formula (VI) is formed:

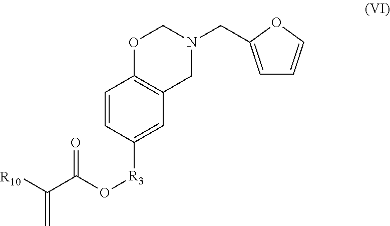

wherein R₃ is a hydrocarbylene group comprising 1 to 10 carbon atoms and R₁₀ is selected from hydrogen and a straight or branched alkyl group comprising 1 to 20 carbon atoms.

When the reagent is epichlorohydrin a second reaction product according to Formula (VII) is formed:

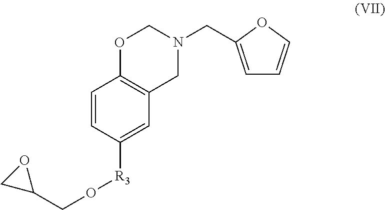

wherein R₃ is a hydrocarbylene having 1 to 10 carbon atoms.

Method of Preparing Benzoxazine Compound (C) According to Formula (III), Wherein at Least One of R₄ and R₈ are R_{C}OH

This method comprises a step of reacting a furfuryl amine, a formaldehyde compound, and a 3,5(hydroxyalkyl)phenolic compound to from a first reaction product. Preferably, the furfuryl amine is present in an amount of about 50 g (51.5 mol), the formaldehyde compound is present in an amount of from about 30.92 g (103 mmol) to 46.38 g (154.5 mmol), and the 3,5-(hydroxyalkyl)phenolic compound is present in an amount of from about 79.36 g (51.5 mmol) to about 87.30 g (56.6 mmol). The reacting step may also be carried out with a molar ratio of furfuryl amine to formaldehyde to the 3,5-(hydroxyalkyl)phenolic compound of from about 1:1:1 to about 1:3:1, or from about 1:1:1 to 1:2:1. In some embodiments, 3,5-(hydroxyalkyl)phenolic compound is 3,5(hydroxymethyl)phenol.

Method of Preparing a Second Reaction Product, Using the First Reaction Product, Benzoxazine Compound (C) According to Formula (III), Wherein at Least One of R₄ and R₅ are R_{C}OH

This method of preparing the second reaction product comprises first preparing the first reaction product, benzoxazine compound (C) according to Formula (III), as set forth above. The first reaction product is then reacted with either a halo-containing epoxide, preferably epichlorohydrin, or a radically polymerizable monomer in the presence of a base catalyst.

Suitable examples of the radically polymerizable monomer may be selected from methacryloyl chloride, methacrylic anhydride, acryloyol chloride, acrylic anhydride, acrylic acid, methacrylic acid, and alkyl anhydrides comprising from 2 to 20 carbon atoms.

Suitable examples of the base catalyst may be selected from dimethylaminopyridine, trimethylamine, 1,8-diazabicyclo[5.4.0]undec-7-ene, 1-methylimidazole and 2-methylimidazole, and triethylamine.

When the reagent is the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms a second reaction product according to Formula (VIII) is formed:

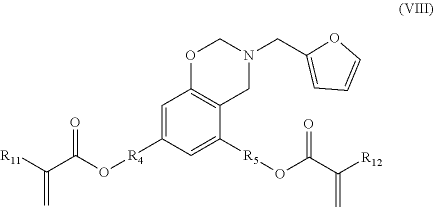

wherein R₄ and R₅ are each independently selected from a hydrocarbylene having 1 to 10 carbon atoms, and R₁₁ and R₁₂ are each independently selected from hydrogen, and straight or branched alkyl group having 1 to 20 carbon atoms.

When the reagent is epichlorohydrin a second reaction product according to Formula (IX) is formed:

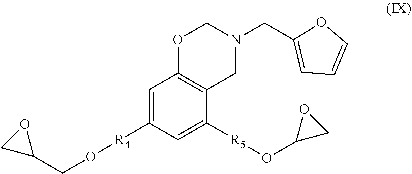

wherein R₄ and R₅ are each independently selected from a hydrocarbylene having 1 to 10 carbon atoms.

### Reaction Product of Benzoxazine and a Reagent

The present disclosure also relates to a compound which is a reaction product prepared from any one of Benzoxazines (A)-(C) and either a halo-containing epoxide, preferably epichlorohydrin, or a radically polymerizable monomer.

The following compounds formed by reaction of any one of benzoxazines (A)-(C) and the reagent may be a liquid at room temperature, where room temperature may be a temperature of about 20° C. to 25° C.

In one embodiment, a reaction product of the Formula (IV) may be formed by reacting a compound of Formula (I) with a radically polymerizable monomer, which may be selected from methacryloyl chloride, methacrylic anhydride, acryloyol chloride, acrylic anhydride, acrylic acid, methacrylic acid, and alkyl anhydrides comprising at least 2-20 carbon atoms:

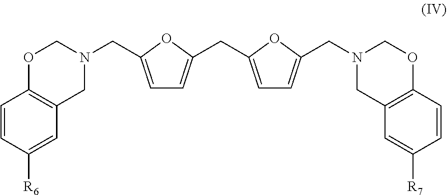

wherein R₆ and R₇ are each independently selected from hydrogen or a group having the Formula (X), and wherein at least one of R₆ and R₇ is a group having the Formula (X):

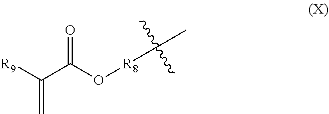

wherein R₈ in Formula (X) is a hydrocarbylene group comprising 1 to 10 carbon atoms, R₉ is selected from hydrogen, and a straight or branched alkyl group having 1 to 20 carbon atoms, and

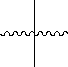

represents the bond attached to the ring carbon of the benzoxazine in Formula (IV).

In some embodiments, R₆ and R₇ may each be the group according to Formula (X).

In one embodiment, a reaction product of Formula (V) may be formed by reacting a compound of the Formula (I) with epichlorohydrin:

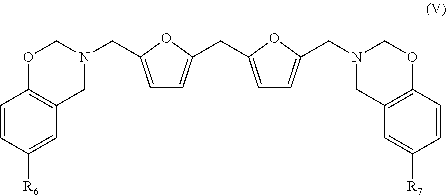

wherein R₆ and R₇ are each independently selected from hydrogen or a group having the Formula (XI), and wherein at least one of R₆ and R₇ is a group having the Formula (XI):

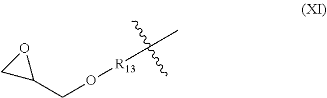

wherein R₁₃ is a hydrocarbylene group comprising 1 to 10 carbon atoms, and

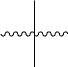

represents the bond attached to the ring carbon of the benzoxazine in Formula (V).

In some embodiments, R₆ and R₇ may each be the group according to Formula (XI).

In one embodiment, a reaction product of the Formula (VI) may be formed by reacting a compound of the Formula (II) with a radically polymerizable monomer which may be selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms:

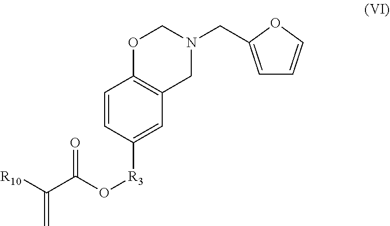

wherein R₃ is a hydrocarbylene group comprising 1 to 10 carbon atoms and R₁₀ is selected from hydrogen and a straight or branched alkyl group comprising 1 to 20 carbon atoms.

In some embodiments, the reaction product according to Formula (VI) may be (3-(furan-2-ylmethyl)-3,4-dihydro-2H-benzo[e][1,3]oxazin-6-yl)methyl methacrylate.

In one embodiment, a reaction product of the Formula (VII) may be formed by reacting a compound of the Formula (II) with epichlorohydrin:

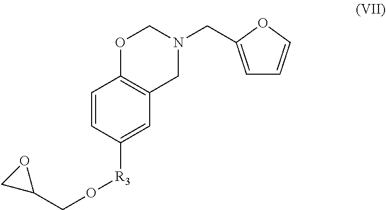

wherein R₃ is a hydrocarbylene group comprising 1 to 10 carbon atoms.

In one embodiment, a reaction product of the Formula (VIII) may be formed by reacting a compound of the Formula (III) with a radically polymerizable monomer which may be selected from methacryloyl chloride, methacrylic anhydride, acryloyol chloride, acrylic anhydride, acrylic acid, methacrylic acid, and alkyl anhydrides having 2 to 20 carbon atoms:

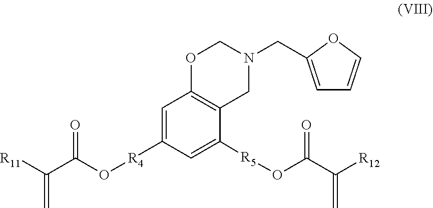

wherein R₄ and R₅ are each independently selected from a hydrocarbylene having 1 to 10 carbon atoms, and R₁₁ and R₁₂ are each independently selected from hydrogen, and straight or branched alkyl groups having 1 to 20 carbon atoms.

In one embodiment, a reaction product of the Formula (IX) may be formed by reacting a compound of the Formula (III) with epichlorohydrin:

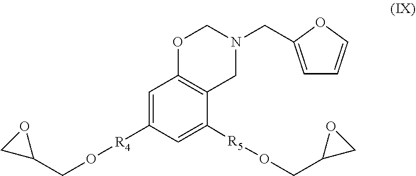

wherein R₄ and R₅ are each independently selected from a hydrocarbylene having 1 to 10 carbon atoms.

### EXAMPLES

### 1.1 Materials

Furfurylamine, phenol, guaiacol, 4-hydroxybenzyl alcohol, paraformaldehyde, chloroform, sodium hydroxide, 4-Dimethylaminopyridine (DMAP), methacrylic anhydride, magnesium sulfate anhydrous, formaldehyde solution (37%), and hydrochloric acid solution (37%) were purchased from Sigma Aldrich; and cardanol was purchased from Cardolite. All chemicals were used as received. The 5,5′-methylenedifurfurylamine (DFDA) was synthesized according the method detailed in literature.¹²

### 1.2 Preparation of Bio-Based Liquid Benzoxazine Monomers

### 1.2.1 Preparation of Asymmetrical DFDA-Based Benzoxazines

Chloroform (50 ml), paraformaldehyde (3.53 g, 116.5 mmol) and DFDA (6 g, 29.1 mmol) were introduced into a round bottomed flask and mixed at room temperature for 30 minutes. After adding different molar ratios of combinations of phenol (e.g. phenol and guaiacol, phenol and cardanol, and guaiacol and cardanol), the various mixtures were stirred at 70° C. and refluxed for 20 hours. The liquid mixtures were first washed with sodium hydroxide solution (0.5 N) and distilled water, then dried using magnesium sulfate, and then the solvent was removed by a rotary evaporator.

¹H-NMR of BZ-DFDA-Phenol/Cardanol (500 MHz, CDCl₃, ppm): δ 6.65-7.14 (m, ArH); 6.04-6.17 (s, furanH); 4.67-5.44 (m, CH═, CH₂═CH—, —O—CH₂—N—); 3.50-4.05 (m, —CH₂-furan-, —N—CH₂Ar); 2.82 (m, —CH₂(CH═)₂); 2.53 (m, —CH₂Ar); 2.03 (s, CH₂CH═); 1.60-1.26 (m, —(CH₂)ₙ—); 0.90 (m, —CH₃).¹³

### 1.2.2 Preparation of Mono-Furan Based Benzoxazine Monomers and Vinyl Ester-Functional Benzoxazine Monomers

1.2.2.1 Preparation of (3-(furan-2-ylmethyl)-3,4-dihydro-2H-benzo[e][1,3]oxazin-6-yl)methanol (BZ-FA-H)

Paraformaldehyde (30.92 g, 103 mmol, 4-hydroxybenzyl alcohol (63.91 g, 51.5 mmol) and furfurylamine (50 g, 51.5 mmol) were introduced into a round bottomed flask and stirred at 80° C. and refluxed for 20 hours. The liquid mixture was first dissolved into ethyl acetate, then it was washed with sodium hydroxide solution (1 N) and distilled water, dried using magnesium sulfate, and then the solvent was removed by rotary evaporator to obtain a yellowish viscous liquid.

(Yield: 60%, Purity: 97%). ¹H-NMR of BZ-FA-H (500 MHz, CDCl₃, ppm): δ 7.43 (s, 1H); 7.13 (d, 1H); 6.99 (d, 1H); 6.80 (d, 1H); 6.35 (s, 1H); 6.25 (s, 1H); 4.89 (s, 2H); 4.58 (s, 2H); 4.02 (s, 2H); 3.93 (s, 2H).

1.2.2.2 Preparation of (3-(furan-2-ylmethyl)-3,4-dihydro-2H-benzo[e][1,3]oxazin-6-yl)methyl methacrylate (BZ-FA-H-MA)

BZ-FA-H (40 g, 127.7 mmol) was dissolved in dichloromethane (30 ml), followed by dropwise addition of methacrylic anhydride (21.6 g, 140.5 mmol) and DMAP (4 g, 32.7 mmol). The mixture was stirred for 20 hours at room temperature, then the solution was washed with saturated sodium bicarbonate solution and distilled water, then dried using magnesium sulfate and the solvent was removed using a rotary evaporator to obtain a yellowish liquid.

(Yield: 78%, Purity: 93%). ¹H-NMR of BZ-FA-H (500 MHz, CDCl₃, ppm): δ 7.43 (s, 1H); 7.15 (d, 1H); 6.99 (d, 1H); 6.80 (d, 1H); 6.35 (s, 1H); 6.25 (s, 1H); 6.13 (d, 1H); 5.56 (s, 1H); 5.10 (s, 2H); 4.89 (s, 2H); 4.03 (s, 2H); 3.91 (s, 2H); 1.97 (s, 3H).

### 1.3 Characterization

Mid-FTIR was used to determine the presence of oxazine rings and other functional groups on the benzoxazines and the methacryloyl-functional benzoxazine using a Thermo Nicolet Nexus 870 FT-IR spectrometer with 32 scans. Differential scanning calorimetry (DSC) was used to observe curing the behavior of benzoxazines at a heating rate of 10° C./min under nitrogen atmosphere. The viscosity of benzoxazine was tested by a TA AR2000 at shear rates of from 0.01 to 100 1/s and employing 40 mm parallel plate geometry. Dynamic mechanical analysis (DMA Q800) was used to study the thermal and mechanical properties of cured benzoxazines with single cantilever geometry and a 2° C./min ramp rate. A TA Q50 TGA was employed to investigate the thermal stability of polybenzoxazines in an argon environment with a 10° C./min ramp rate. A servo-hydraulic Instron 8872 with a 1000 N load cell was used to carry out tensile tests on cured benzoxazine materials.

### 1.4 Preparation of Cured Materials

All cured benzoxazines were prepared by pouring liquid benzoxazine monomer into a rectangular silicone mold with dimensions of 17.5 mm×13 mm×3 mm for Dynamic Mechanical Analysis (DMA). The following curing conditions of DFDA-based benzoxazines were employed: 180° C. for 1 hour; 200° C. for 2 hours; 220° C. for 2 hours; 240° C. for 1 hour, and 260° C. for 1 hour. The sample of BZ-FA-H in the silicone mold was heated stepwise at 180° C. for 2 hours; 200° C. for 2 hours; and 220° C. for 2 hours. For methacryloyl-functionalized benzoxazine, since the CH₂—O—C═O linkage cannot endure a high temperature curing process and methyl methacrylate is able to photo-polymerize, BZ-FA-H-MA was cured under UV light at 80° C. for 3 hours, and was then thermally cured with the same procedure as was used for BZ-FA-H for oxazine ring-opening polymerization.

### Results and Discussion

### 1.4.1 Asymmetrical DFDA Based Benzoxazines

Except for BZ-DFDA-Phenol/Cardanol (90:10), the rest of the tested benzoxazine blends were liquid at room temperature as the content of cardanol was increased. The viscosity of BZ-DFDA-Phenol/Cardanol (80:20) was measured to be 52 Pa·s at 25° C. and 3 Pa·s at 50° C., which are convenient viscosities for molding processes.

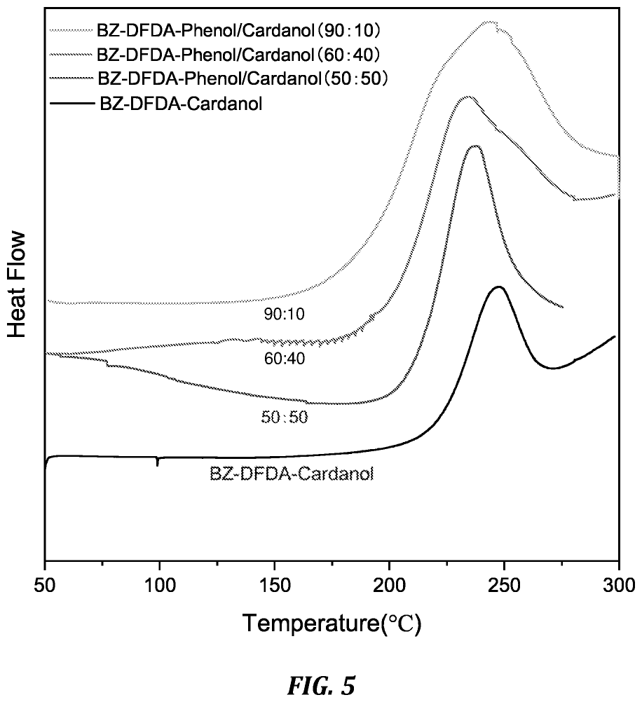

Curing behaviors of different ratios of BZ-DFDA-Phenol/Cardanol benzoxazine blends were observed and depicted in DSC curves. Based on FIG. 5 it is apparent that BZ-DFDA-Cardanol showed an onset temperature of polymerization at 206° C. BZ-DFDA-Cardanol required more heat to start the curing reaction because long chains connected to the benzene rings restricted the mobility of the monomers. As the ratio of phenol increased, a lower onset temperature was obtained. As a result, BZ-DFDA-Phenol/Cardanol benzoxazine blends can achieve onset temperatures between 150° C. and 190° C., similar to typical benzoxazines.

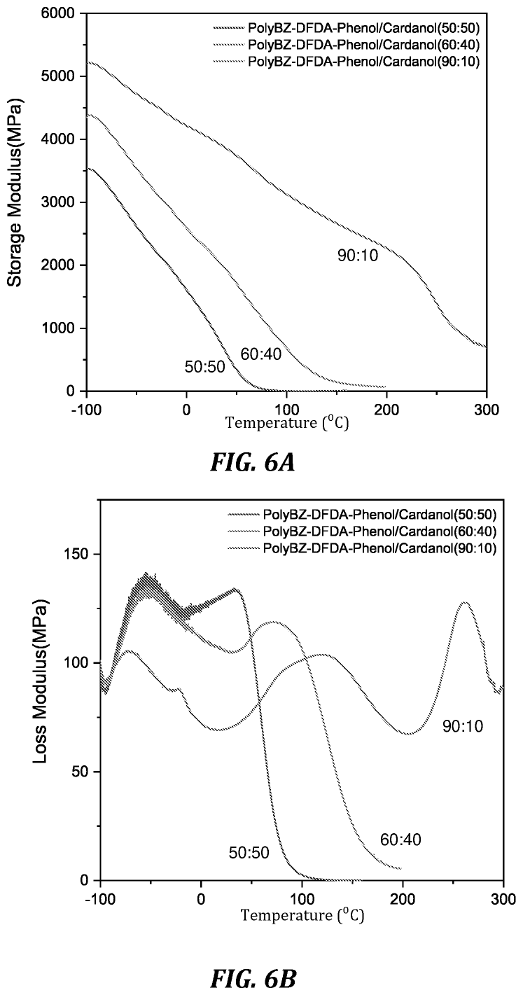

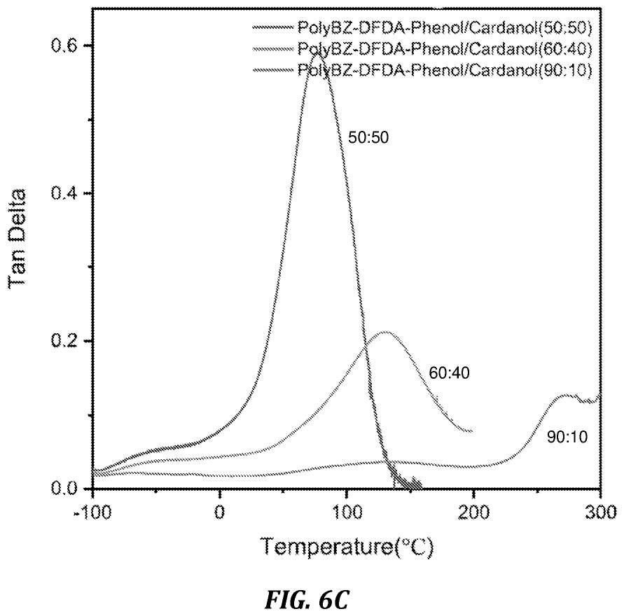

Thermal and mechanical properties of PolyBZ-DFDA-Phenol/Cardanol were explored by DMA and the data is shown in FIGS. 6A-6C. Storage modulus, loss modulus, and tan delta were plotted for each asymmetrical benzoxazine blend separately. As these results show, a higher content of cardanol in the network lessens the storage modulus and T_{g} by decreasing the crosslink density. Relatively lower cardanol contents showed no obvious effect on the network. For example, the T_{g} of PolyBZ-DFDA-Phenol/Cardanol (90:10), based on loss modulus reached to 260° C., which is very close to pure DFDA-based polybenzoxazines. However, 40% and 50% contents of cardanol in the network weakened the degree of polymeric system packing and achieved T_{g}'s of 35° C. and 72° C., respectively.

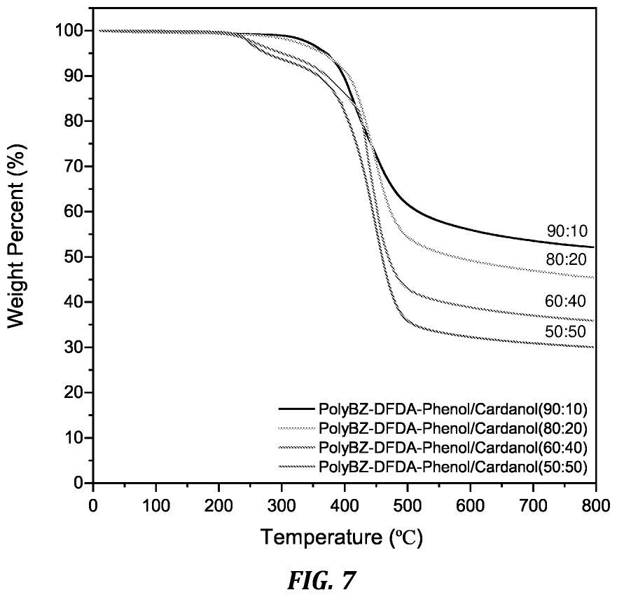

Thermal degradation and stability of the blends were studied by TGA. From the thermograms of FIG. 7, char yields were 54.3% for Phenol/Cardanol (90:10), 45.5% for Phenol/Cardanol (80:20), 35.9% for Phenol/Cardanol (60:40), and 31.0% for Phenol/Cardanol (50:50). The impact of incorporating cardanol on thermal stability is apparent. Further, the addition of cardanol lowered the decomposition temperature-systems containing 10% and 20% cardanol exhibited a degradation temperature of about 320° C. When the content of cardanol was increased to 40% in the network, the polymer started to lose weight at 250° C. because when more fatty chains are involved in the network, this leads to lower crosslink density systems with less thermal stability.

### 1.4.2 Mono-Furan Based Benzoxazine Monomers and Vinyl Ester-Functional Benzoxazine Monomers

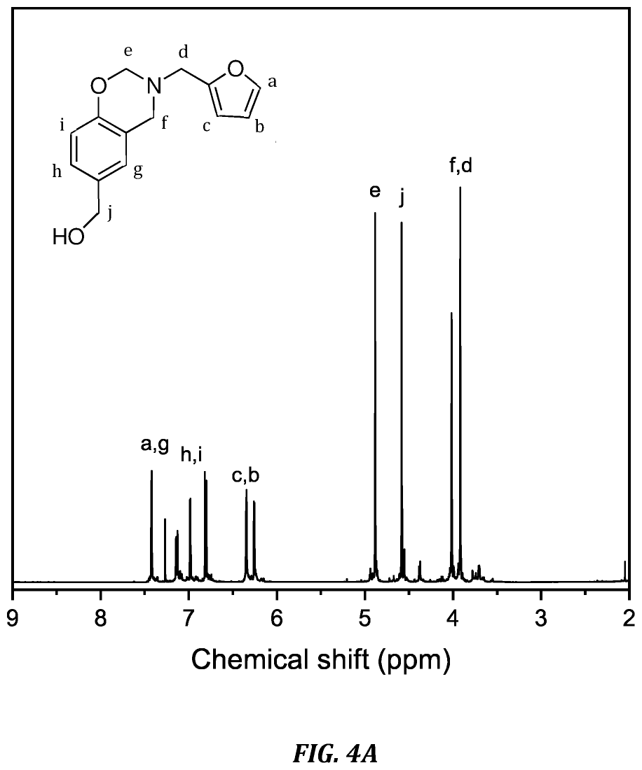

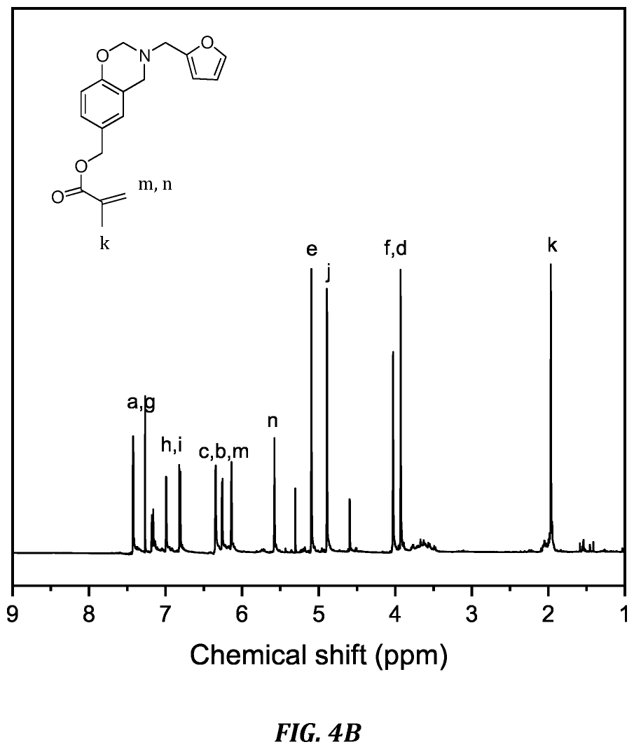

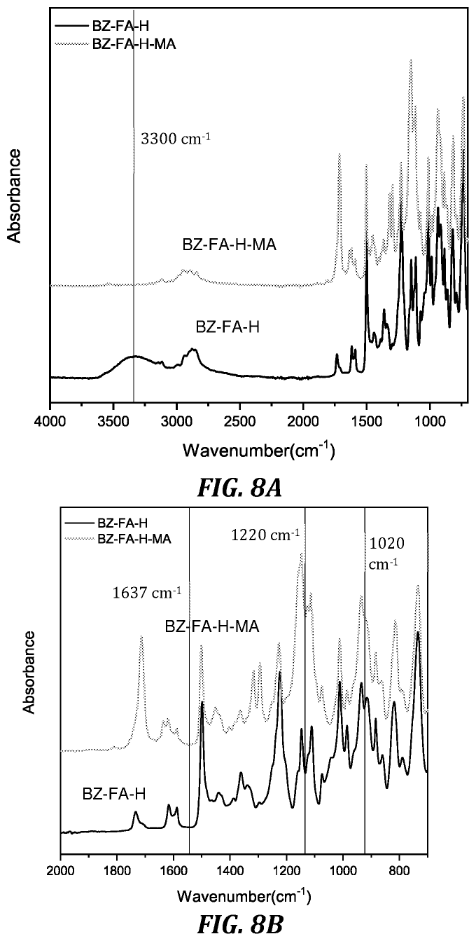

The structures of the novel benzoxazine monomer and methacryloyl-functional benzoxazine were confirmed by ¹H NMR. The ¹H NMR spectra are shown in FIG. 4. For both BZ-FA-H and BZ-FA-H-MA, characteristic peaks at 4.02 and 4.89 ppm are assigned to the protons of the oxazine rings —Ar—CH₂—N— and —O—CH₂—N—, respectively. Furan ring protons give rise to the resonances at 6.25 and 6.35 ppm. Comparing (a) to (b) after the esterification reaction, the resonance of BZ-FA-H that corresponds to the methylol group centered at 4.58 ppm shifted to 4.89 ppm in the spectrum of BZ-FA-H-MA. In addition, the ¹H NMR elemental analysis of benzoxazines shown in the experimental section correlates well with the theoretical values calculated for the novel benzoxazine molecular structures. The Mid-FTIR spectra shown in FIG. 8 are also consistent with the structure of benzoxazines. The characteristic absorption peak for the hydroxy group was observed at about 3300 cm⁻¹ in BZ-FA-H and disappeared in BZ-FA-H-MA. The presence of the C═C of the methacrylate groups of BZ-FA-H-MA was confirmed by the peak absorption at 1637 cm⁻¹. In addition, the absorption peaks at 1220 cm⁻¹ for Ar—C—O stretch and 1020 cm⁻¹ for asymmetric stretching of C—N—C, suggest that the benzene ring is fused to an oxazine ring.

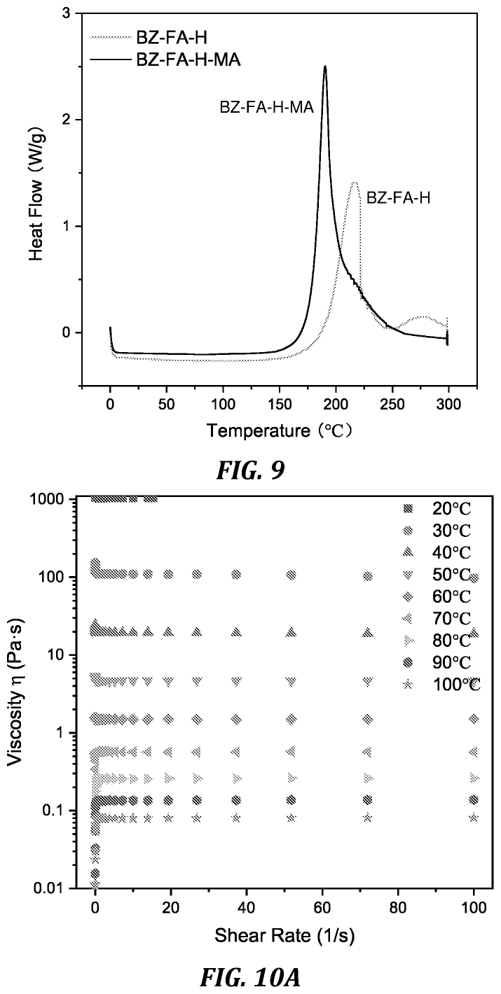

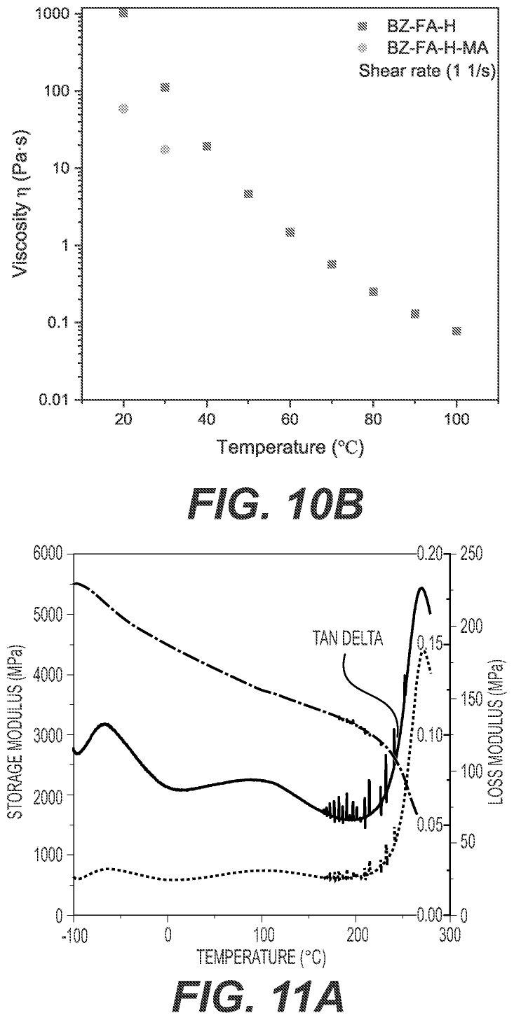

Since the novel benzoxazines are liquid at room temperature, there are no endothermic peaks of melting points in the DSC curves shown in FIG. 9. The exothermic peaks for the BZ-FA-H and BZ-FA-H-MA monomers were observed in the range of 160-250° C. Based on previous studies from other groups, ring-opening polymerization of benzoxazines typically exhibit exothermic peaks around 200-280° C. The bio-based benzoxazine monomers showed similar exothermic behaviors at the lower temperature stage, which is beneficial for industrial processing. In addition, the heat of the polymerization reactions for both BZ-FA-H and BZ-FA-H-MA are 215.6 and 343.6 J/g, respectively. BZ-FA-H-MA requires more energy for a curing reaction than BZ-FA-H because it contains two reactions: the ring-opening of oxazine groups and the vinyl polymerization of methacrylate. The viscosity of bio-based benzoxazines versus temperature was measure using a rheometer with shear rates that ranged from 0.01 to 100 1/s. All liquid benzoxazines showed Newtonian fluid behavior in isothermal measurements. As shown in FIG. 10, the viscosity of BZ-FA-H reached 1000 Pa·s at 20° C. and decreased to 100 Pa·s at 30° C., which is enough of a decrease to be able to process molding materials. The viscosity of BZ-FA-H-MA was 17 Pa·s at 30° C., and it exhibited more flowability at room temperature due to the elimination of the hydroxy groups and the appearance of acrylates in the structure. Compared to typical solid benzoxazines, all bio-based benzoxazines have good processability for liquid molding transfer.

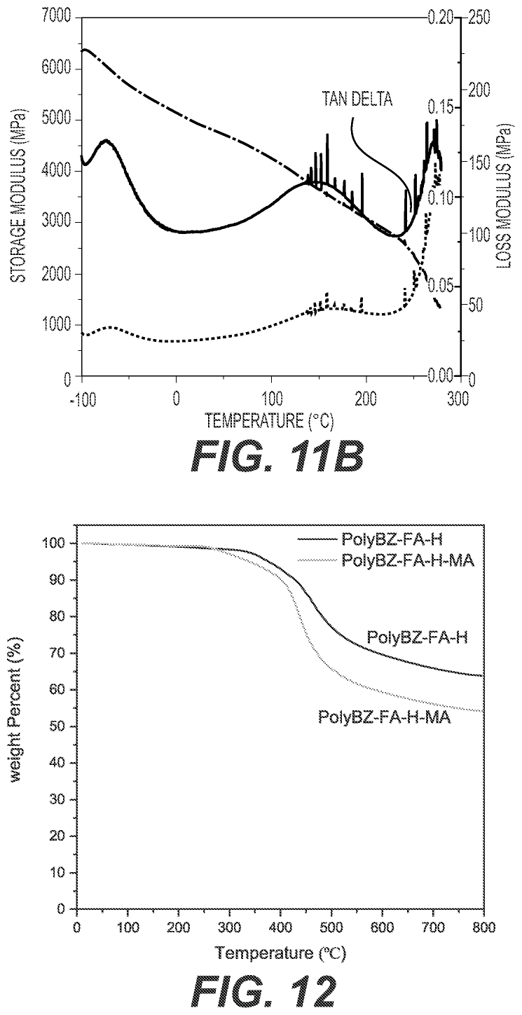

DMA was used to investigate the dynamic mechanical and thermal properties of bio-based polybenzoxazines. FIG. 11 presents the storage modulus and loss modulus curves of the cured materials, which curves were obtained using a ramp rate of 2° C./min. The storage modulus of the polybenzoxazine and methacryloyl-polybenzoxazine was above 4 GPa at room temperature, as is expected in terms of high crosslink network and furan groups. The glass transition temperature (T_{g}) of the polybenzoxazine and methacryloyl-polybenzoxazine showed similar behavior in the scanning. The PolyBZ-FA-H with a 220° C. post curing temperature resulted in a T_{g} of 269° C. based on loss modulus; the addition polymerization of acrylates groups of PolyBZ-FA-H-MA did not affect the network and a 269° C. T_{g} was also obtained with acrylates in formulations.

The thermal stabilities of PolyBZ-FA-H and PolyBZ-FA-H-MA were observed by Thermogravimetric Analysis (TGA). The curves in FIG. 12 indicate that PolyBZ-FA-H and PolyBZ-FA-H-MA possess char yields of 64% and 54% at 800° C., respectively. PolyBZ-FA-H started to lose mass weight at temperatures of from about 350° C. The decomposition temperature of PolyBZ-FA-H-MA, however, was lower than that of PolyBZ-FA-H because the —CH₂—CH₂— linkage from self-polymerized methyl methacrylate was not able to withstand high temperatures.

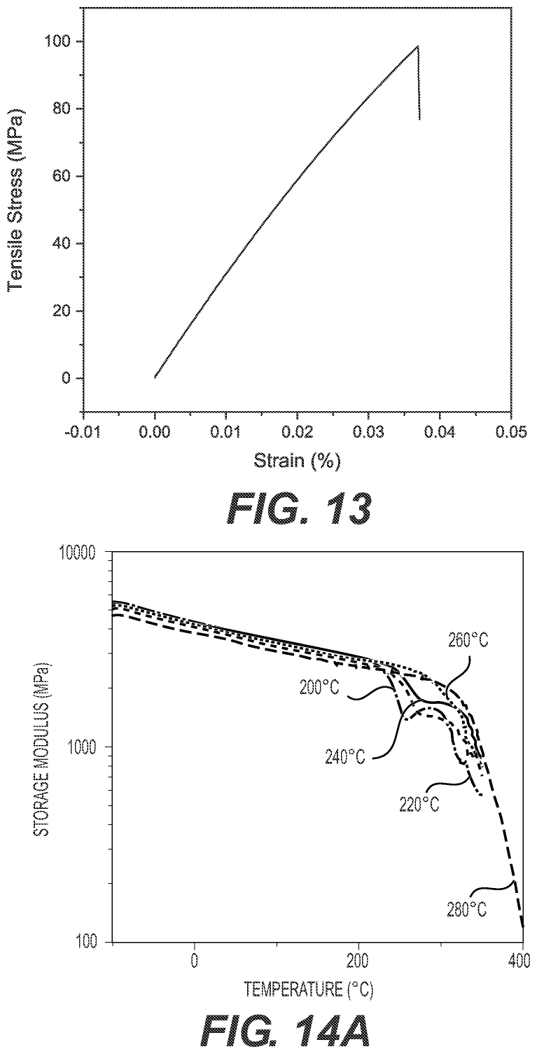

Tensile test measurements were conducted by Instron, and linear elastic behaviors of mono-functional polybenzoxazine were observed. The stress-strain behavior is shown in FIG. 13. The Young's modulus of PolyBZ-FA-H was 4.95 GPa, which was obtained using the initial slope of the tensile stress-strain curve. Compared to the 1.0% elongation of B-m with a 44 MPa stress and 1.3% elongation of B-a with a 64 MPa stress, PolyBZ-FA-H failed at 3.7% elongation with a 96 MPa tensile stress, indicating that it provided a significantly better performance than that of BPA-based polybenzoxazines.¹⁴ Therefore, PolyBZ-FA-H not only possesses high tensile modulus but also exhibits high elongation at break and tensile strength.

### BZ-FA-H

### Supplementary Tests

1.1 Fracture Toughness

TABLE 1

| K_{1c} (MPa*m^{1/2}) | G_{1c} (J/m²) |
|---|---|
| 0.89 ± 0.06 | 141.56 ± 17.52 |

Table 1 shows the K_{1c} and G_{1c} of PolyBZ-FA-H from a fracture toughness test.

The fracture toughness test was performed at room temperature using an Instron Model 8872. Fracture toughness single-edge-notch bending (SENB) specimens were prepared and tested following the procedure of ASTM 5045-99. The SENB specimens had dimensions of 44×10×5 mm. A crack was initiated at the bottom of the notch using a sharp razor blade. An average of five specimens were tested at a crosshead speed of 10 mm/min. The sample was tested based on a 220° C. post curing temperature. The K_{1c} and G_{1c} values that were obtained are compatible with most of thermosets. K_{1c} may be defined as the plane strain critical stress intensity factor. G₁c may be defined as the plane strain critical strain energy release rate.

1.2 DMA Tests of PolyBZ-FA-H Based on a Different Post Curing Temperature

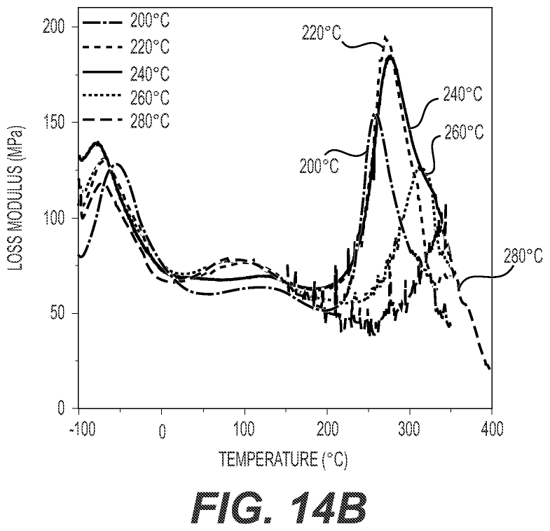

It was found that PolyBZ-FA-H post cured at a different temperature exhibited a different thermal performance due to a change in the extent of the furan reaction that resulted from the different curing temperature. DMA was used to test their performance. The curves in FIGS. 14A-14B indicate that PolyBZ-FA-H post cured at 200° C., 220° C., 240° C., 260° C., and 280° C., possessed Tgs, based on loss modulus, of 259° C., 269° C., 274° C., 314° C., and 338° C., respectively. This demonstrates that the Tg of PolyBZ-FA-H can be controlled by selection of appropriate post curing procedures such that the material can be customized for different uses.

### Other Mono-Furan Based Benzoxazines

2.1 Characterization of Other Mono-Furan Based Benzoxazines

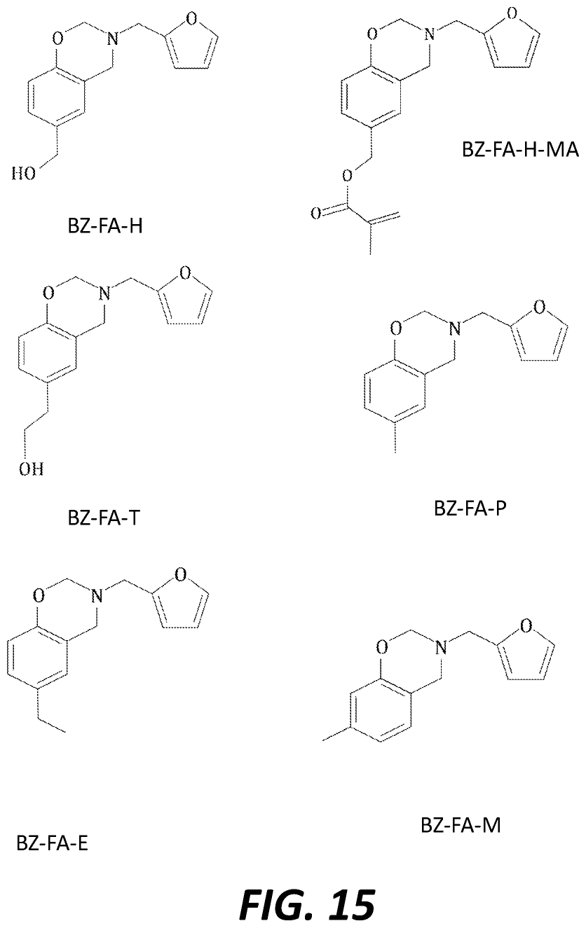

Except for BZ-FA-H, FIG. 15 shows mono-furan based benzoxazine monomers that were synthesized using different types of phenolic compounds. Table 2 summarizes the data of the DSC thermograms of these mono-furan based benzoxazines. BZ-FA-H is liquid at room temperature. Other mono-furan based benzoxazines were able to crystallize and exhibited melting points (Tm) ranging from 40° C. to 70° C., which makes them easily processable in various useful applications. The temperature at which onset of polymerization took place for these mono-furan based benzoxazines was in the range of about 140° C. to about 220° C. This is significantly lower than 200° C. to 250° C. exothermic peaks of ring-opening polymerization from other types of benzoxazines.

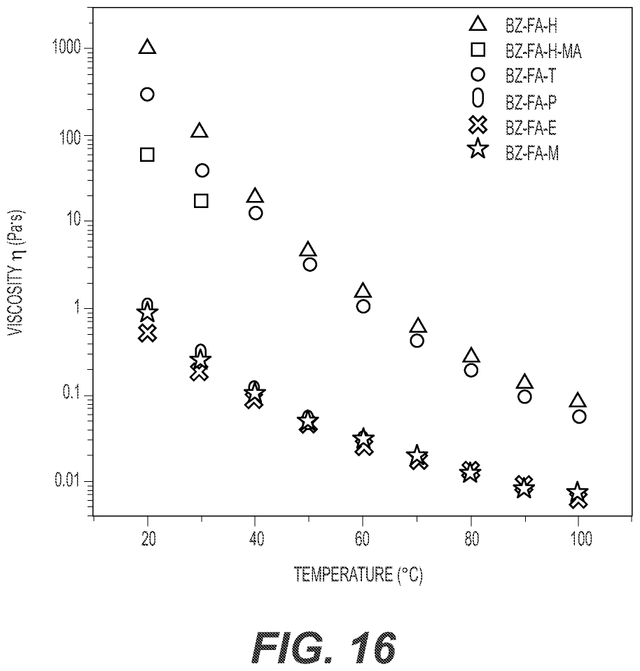

As shown in FIG. 16, the viscosity of BZ-FA-H reached 1000 Pa·s at 20° C. and decreased to 100 Pa·s at 30° C., which was enough of a decrease to be able to process molding materials. The viscosity of BZ-FA-H-MA was 17 Pa·s at 30° C., and it exhibited more flowability at room temperature due to the elimination of the hydroxy groups and the appearance of acrylates in the structure. Compared to typical solid benzoxazines, all bio-based benzoxazines have good processability for liquid molding transfer. Other mono-furan based benzoxazines show subcooling liquid behavior, because it takes time for them crystallize at room temperature, which results in a tested viscosity at 20° C. Since BZ-FA-E, BZ-FA-P, and BZ-FA-M don't have hydroxy groups fused to the benzene ring, the viscosity of these monomers is much lower than the viscosity of BZ-FA-H. BZ-FA-T, which has a similar structure to that of BZ-FA-H, shows rheological behavior that is consistent with BZ-FA-H.

TABLE 2
DSC data of mono-furan based benzoxazines

| Benzoxazine | Tₘ (° C.) | Tₒₙₛₑₜ (° C.) | Tₘₐₓ (° C.) |
|---|---|---|---|
| BZ-FA-H | — | 174 | 218 |
| | Liquid at Room | | |
| | Temperature | | |
| BZ-FA-H-MA | 60 | 148 | 190 |
| BZ-FA-T | 69 | 159 | 201 |
| BZ-FA-P | 53 | 214 | 235 |
| BZ-FA-E | 47 | 180 | 232 |
| BZ-FA-M | 66 | 217 | 251 |

2.2 Thermal and Mechanical Properties of Other Mono-Furan Based Benzoxazines

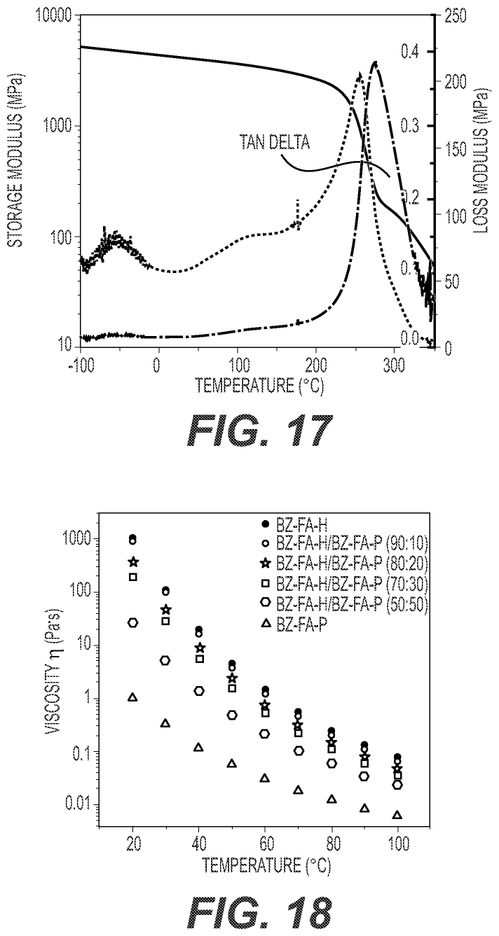

PolyBZ-FA-M as an example of a mono-furan based benzoxazine without hydroxy and acrylate groups. Post curing of PolyBZ-FA-M at 220° C. resulted in a T_{g} of 255° C. based on loss modulus (as shown in FIG. 17). This Tg is lower than that of PolyBZ-FA-H because of the lack of hydrogen bonds, However, the PolyBZ-FA-M performs much better when compared with other polybenzoxazines.

### BZ-FA-H/BZ-FA-P Copolymer System

3.1 Viscosity of the BZ-FA-H/BZ-FA-P Copolymer System

Since BZ-FA-P has very low viscosity compared with BZ-FA-H, BZ-FA-P can be used as a diluent to lower the viscosity of BZ-FA-H. For this copolymer mixture, the viscosity decreased linearly with an increasing molar ratio of BZ-FA-P to BZ-FA-H as shown in FIG. 18. The viscosity of BZ-FA-H/BZ-FA-P (50:50 molar ratio) reached about 1 Pa·s at room temperature, which makes it suitable for liquid molding transfer and preparation of RTM composites at room temperature without external heating.

3.2 Thermal and Mechanical Properties of the PolyBZ-FA-H/BZ-FA-P Copolymer System

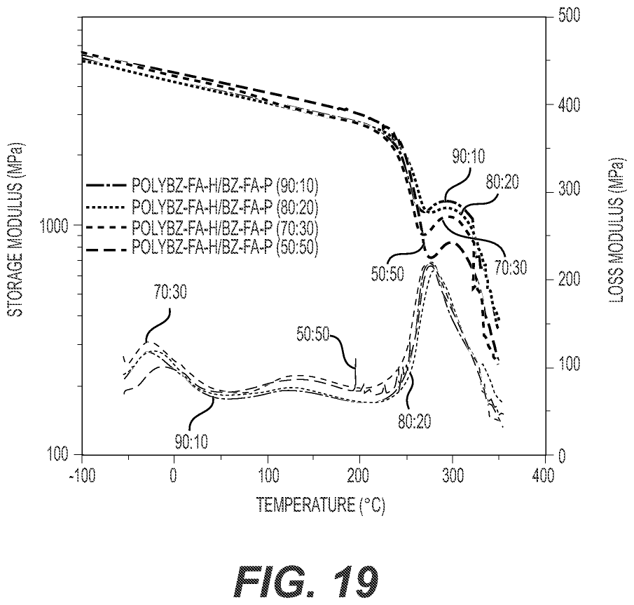

FIG. 19 presents the storage modulus and loss modulus curves of the copolymer system, the storage modulus of all mixtures was above 4 GPa at room temperature. The Tg of the all combinations showed similar behavior in the scanning in terms of similar structures of BZ-FA-H and BZ-FA-P. The PolyBZ-FA-H/BZ-FA-P copolymers exhibit Tg ranged from 261 to 268° C. based on loss modulus; it is obvious to see that combining with BZ-FA-P has little influence on the polyBZ-FA-H network.

### Di-Furan Based Benzoxazine BZ-DFDA-H

4.1 Characterization of BZ-DFDA-H

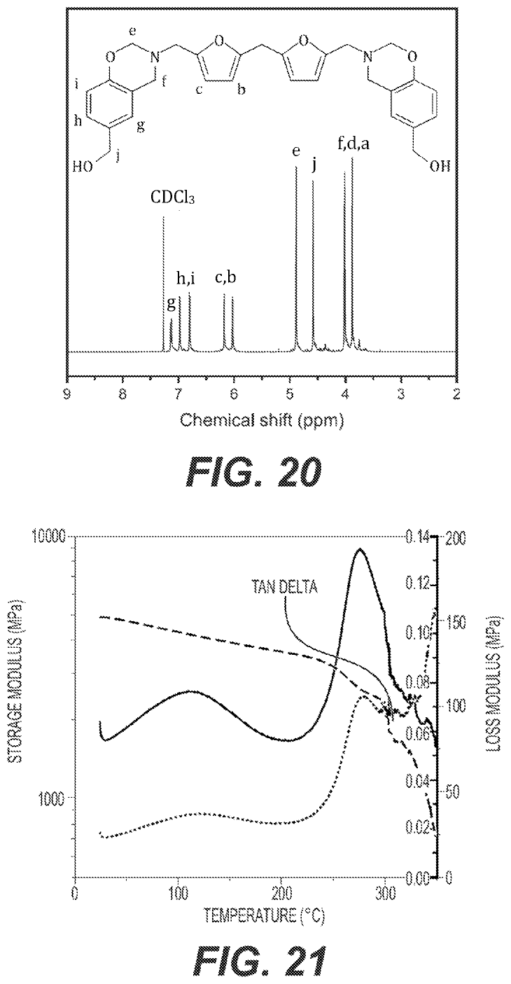

The structure of BZ-DFDA-H was confirmed by ¹H NMR, the resonance peaks at 4.10 ppm and 5.03 ppm corresponds to the protons of the oxazine rings —Ar—CH₂—N— and —OCH₂—N— in FIG. 20, respectively. The proton of the furan rings gives rise to the characteristic peaks at 6.03 ppm and 6.09 ppm.

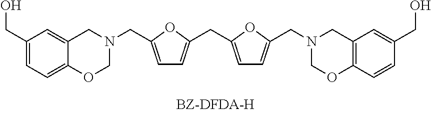 [SMILES: OCc1ccc2c(c1)CN(Cc1ccc(Cc3ccc(CN4COc5ccc(CO)cc5C4)o3)o1)CO2]

4.2 Thermal and Mechanical Properties of PolyBZ-DFDA-H

PolyBZ-DFDA-H performs similarly to other mono-furan benzoxazines in DMA scanning tests. For example, the storage modulus at room temperature reached about 4 GPa. The Tg value of polyBZ-DFDA-H obtained from the loss modulus was 275° C., based on a 220° C. post curing temperature, which was slightly higher than that of polyBZ-FA-H due to the higher crosslink density of the di-functional system. See FIG. 21.

### 2. Conclusion

A series of liquid bio-based benzoxazine monomers, derived from furfurylamine and phenolic compounds from natural extraction, were synthesized with the use of asymmetrical structures and the addition of methacrylate groups. ¹H NMR and FTIR characterizations were used to confirm the structures of the benzoxazines. All benzoxazine precursors were viscous liquids at room temperature and, as such, easy to process for multiple applications. In addition, bio-based polybenzoxazine systems showed good thermal stability and high T_{g} with a high renewable content.

### REFERENCES

1. Wang, Y. X. & Ishida, H. Synthesis and properties of new thermoplastic polymers from substituted 3,4-dihydro-2H-1,3-benzoxazines. Macromolecules 33, 2839-2847 (2000).
2. Lligadas, G., Tüzün, A., Ronda, J. C., Galià, M. & Cádiz, V. Polybenzoxazines: New players in the bio-based polymer arena. Polym. Chem. 5, 6636-6644 (2014).
3. Ghosh, N. N., Kiskan, B. & Yagci, Y. Polybenzoxazines-New high performance thermosetting resins: Synthesis and properties. Prog. Polym. Sci. 32, 1344-1391 (2007).
4. Holly, F. W. & Cope, A. C. Condensation Products of Aldehydes and Ketones with o-Aminobenzyl Alcohol and o-Hydroxybenzylamine. J. Am. Chem. Soc. 66, 1875-1879 (1944).
5. Takeichi, T., Kawauchi, T. & Agag, T. High Performance Polybenzoxazines as a Novel Type of Phenolic Resin. Polym. J. 40, 1121-1131 (2008).
6. Ishida, H. & Agag, T. Handbook of Benzoxazine Resins. Handb. Benzoxazine Resins 740480 (2011). doi:10.1016/C2010-0-66598-9
7. Dumas, L., Bonnaud, L., Olivier, M., Poorteman, M. & Dubois, P. Eugenol-based benzoxazine: From straight synthesis to taming of the network properties. J. Mater. Chem. A 3, 6012-6018 (2015).
8. Puchot, L. et al. Breaking the symmetry of dibenzoxazines: A paradigm to tailor the design of bio-based thermosets. Green Chem. 18, 3346-3353 (2016).
9. Liu, Y. L. & Chou, C. I. High performance benzoxazine monomers and polymers containing furan groups. J. Polym. Sci. Part A Polym. Chem. 43, 5267-5282 (2005).
10. Sini, N. K., Bijwe, J. & Varma, I. K. Renewable benzoxazine monomer from Vanillin: Synthesis, characterization, and studies on curing behavior. J. Polym. Sci. Part A Polym. Chem. 52, 7-11 (2014).
11. Jin, L., Agag, T., Yagci, Y. & Ishida, H. Methacryloyl-functional benzoxazine: Photopolymerization and thermally activated polymerization. Macromolecules 44, 767-772 (2011).
12. Holfinger, M. S., Conner, A. H., Holm, D. R. & Hill, C. G. Synthesis of Difurfuryl Diamines by the Acidic Condensation of Furfurylamine with Aldehydes and Their Mechanism of Formation. J. Org. Chem. 60, 1595-1598 (1995).
13. Shukla, S., Mahata, A., Pathak, B. & Lochab, B. Cardanol benzoxazines-interplay of oxazine functionality (mono to tetra) and properties. RSC Adv. 5, 78071-78080 (2015).
14. Ishida, H. & Allen, D. J. Physical and mechanical characterization of near-zero shrinkage polybenzoxazines. J. Polym. Sci. Part B Polym. Phys. 34, 1019-1030 (1996).

1. A benzoxazine compound selected from A)-C):
A) a benzoxazine compound according to Formula (I): 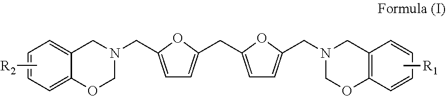
wherein R₁ and R₂ are each independently selected from hydrogen, a straight or branched alkyl group having 1 to 30 carbon atoms, a straight or branched alkenyl group having 5 to 30 carbon atoms, a straight or branched alkoxy group having 5 to 30 carbon atoms, or R_{A}OH, wherein R_{A} is a hydrocarbylene comprising 1 to 10 carbon atoms;
B) a benzoxazine compound according to Formula (II): 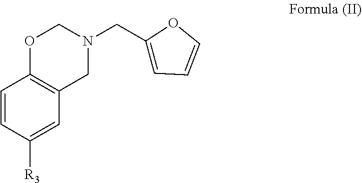
wherein R₃ is selected from a straight or branched alkyl group having 1 to 10 carbon atoms, a straight or branched alkenyl group having 5 to 30 carbon atoms, or a straight or branched chain alkoxy group having 5 to 30 carbon atoms, or R_{B}OH, wherein R_{B} is a hydrocarbylene comprising 1 to 10 carbon atoms; and
C) a benzoxazine compound according to Formula (III): 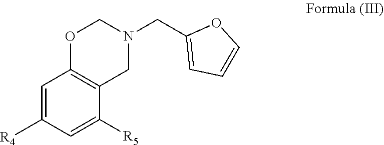
wherein R₄ and R₅ are each independently selected from a straight or branched alkyl group having 5 to 10 carbon atoms, a straight or branched alkenyl group having 5 to 30 carbon atoms, a straight or branched chain alkoxy group having 5 to 30 carbon atoms, or R_{C}OH, wherein R_{C} is a hydrocarbylene having 1 to 10 carbon atoms.

2. The benzoxazine compound of claim 1, wherein the benzoxazine compound is a compound of the Formula (I).

3. The benzoxazine compound of claim 2, wherein R₁ has from 7 to 25 carbon atoms.

4. The benzoxazine compound of claim 2, wherein R₁ is an alkenyl group.

5. The benzoxazine compound of claim 2, wherein each of R₁ and R₂ is selected from hydrogen and R_{A}OH, and at least one of R₁ and R₂ is R_{A}OH.

6. The benzoxazine compound of claim 1, wherein the benzoxazine compound is a compound according to the Formula (II).

7. The benzoxazine compound of claim 6, wherein R₃ is R_{B}OH.

8. The benzoxazine compound of claim 1, wherein the benzoxazine compound is a compound according to Formula (III).

9. The benzoxazine compound of claim 8, wherein R₄ and R₅ are each R_{C}OH.

10. A reaction product prepared by the reaction of:
i) a benzoxazine compound selected from the group consisting of:
a) the benzoxazine compound according to Formula (I): 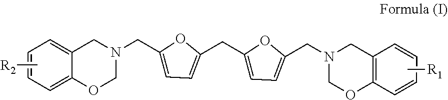
wherein R₁ and R₂ are selected from hydrogen and R_{A}OH, and at least one of R₁ and R₂ is R_{A}OH, wherein R_{A} is a hydrocarbylene comprising 1 to 10 carbon atoms;
b) the benzoxazine compound according to the Formula (II) 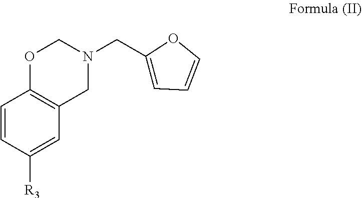
wherein R₃ is selected from a straight or branched alkyl group having 1 to 10 carbon atoms, a straight or branched alkenyl group having 5 to 30 carbon atoms, or a straight or branched chain alkoxy group having 5 to 30 carbon atoms, or R_{B}OH, wherein R_{B} is a hydrocarbylene comprising 1 to 10 carbon atoms; and
c) the benzoxazine compound of the Formula (III) 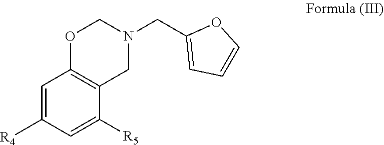
wherein R₄ and R₅ are each R_{C}OH; and
ii) a reagent selected from one of the following:
a) a halo-containing epoxide which is preferably epichlorohydrin; and
b) a radically polymerizable monomer.

11. The reaction product of claim 10, wherein the reaction product is prepared from the benzoxazine compound according to Formula (I): 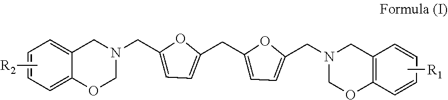
wherein R₁ and R₂ are selected from hydrogen and R_{A}OH, and at least one of R₁ and R₂ is R_{A}OH, wherein R_{A} is a hydrocarbylene comprising 1 to 10 carbon atoms.

12. The reaction product of claim 11, wherein the reagent is the radically polymerizable monomer and is selected from methacryloyl chloride, methacrylic anhydride, acryloyol chloride, acrylic anhydride, acrylic acid, methacrylic acid, and alkyl anhydrides comprising at least 2-20 carbon atoms, and the reaction product has a Formula (IV): 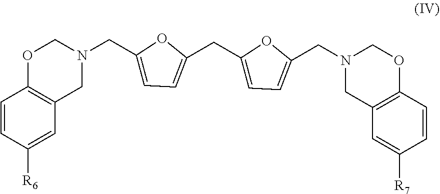
wherein R₆ and R₇ are each independently selected from hydrogen or a group having the Formula (X), and at least one of R₆ and R₇ is a group having the Formula (X): 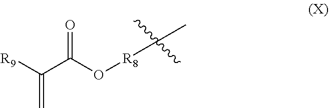
wherein R₈ in Formula (X) is a hydrocarbylene group comprising 1 to 10 carbon atoms, R₉ is selected from hydrogen, and a straight or branched alkyl group having 1 to 20 carbon atoms, and 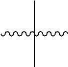
represents the bond attached to the ring carbon of the benzoxazine group in Formula (IV).

13. The reaction product of claim 12, wherein R₆ and R₇ are each the group according to Formula (X).

14. The reaction product of claim 11, wherein the reagent is epichlorohydrin and the reaction product has a Formula (V): 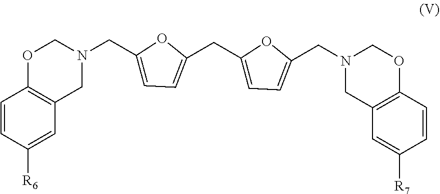
wherein R₆ and R₇ are each independently selected from hydrogen or a group having the Formula (XI), and at least one of R₆ and R₇ is a group having the Formula (XI): 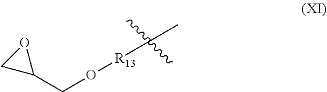
wherein R₁₃ is a hydrocarbylene group comprising 1 to 10 carbon atoms, and 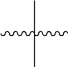
represents the bond attached to the ring carbon of the benzoxazine group in Formula

15. The reaction product of claim 14, wherein R₆ and R₇ are each the group according to Formula (XI).

16. The reaction product of claim 10, wherein the reaction product is prepared from the benzoxazine compound of the Formula (II): 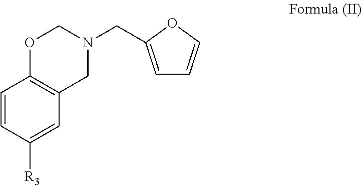
wherein R₃ is selected from a straight or branched alkyl group having 1 to 10 carbon atoms, a straight or branched alkenyl group having 5 to 30 carbon atoms, or a straight or branched chain alkoxy group having 5 to 30 carbon atoms, or R_{B}OH, wherein R_{B} is a hydrocarbylene comprising 1 to 10 carbon atoms.

17. The reaction product of claim 16, wherein the reagent is the radically polymerizable monomer and is selected from methacryloyl chloride, methacrylic anhydride, methyl methacrylate, methacrylic acid, acryloyol chloride, acrylic anhydride, acrylic acid and alkyl anhydrides comprising from 2 to 20 carbon atoms and the reaction product has a Formula (VI): 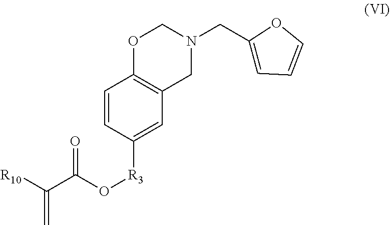
wherein R₃ is a hydrocarbylene group comprising 1 to 10 carbon atoms and R₁₀ is selected from hydrogen and a straight or branched alkyl group comprising 1 to 20 carbon atoms.

18. The reaction product of claim 16, wherein the reaction product is (3-(furan-2-ylmethyl)-3,4-dihydro-2H-benzo[e][1,3]oxazin-6-yl)methyl methacrylate.

19. The reaction product of claim 16, wherein the reagent is epichlorohydrin and the reaction product has a Formula (VII): 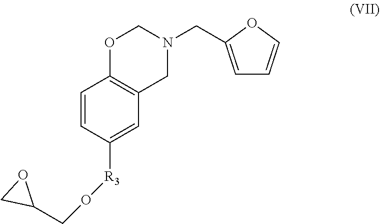
wherein R₃ is a hydrocarbylene group comprising 1 to 10 carbon atoms.

20. The reaction product of claim 10, wherein the compound is a reaction product prepared from a compound of the Formula (III).

21. The reaction product of claim 20, wherein the reagent is the radically polymerizable monomer which is selected from methacryloyl chloride, methacrylic anhydride, acryloyol chloride, acrylic anhydride, acrylic acid, methacrylic acid, and alkyl anhydrides having 2 to 20 carbon atoms and the reaction product as a Formula (VIII): 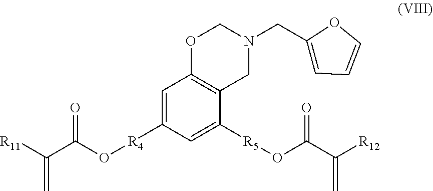
wherein R₄ and R₅ are each independently selected from a hydrocarbylene having 1 to 10 carbon atoms, and R₁₁ and R₁₂ are each independently selected from hydrogen, and straight or branched alkyl groups having 1 to 20 carbon atoms.

22. The reaction product of claim 20, wherein the reagent is epichlorohydrin and the reaction product has a Formula (IX): 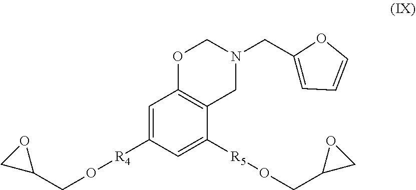
wherein R₄ and R₈ are each independently selected from a hydrocarbylene having 1 to 10 carbon atoms.

23. (canceled)

24. A method of forming the benzoxazine compound of the Formula (I) of claim 5, comprising a step of reacting a difurfuryldiamine, a formaldehyde compound, a phenolic compound, and a cardanol compound.

25-39. (canceled)

40. A method of forming a benzoxazine reaction product comprising steps of:
a) forming a first reaction product as claimed in claim 24; and
b) reacting a first reaction product with a reagent selected from one of the following:
i) a halo-containing epoxide which is preferably epichlorohydrin; and
ii) a radically polymerizable monomer;
in the presence of a base catalyst.

41-59. (canceled)

60. A benzoxazine compound selected from the group consisting of compounds of the formulae A)-F):

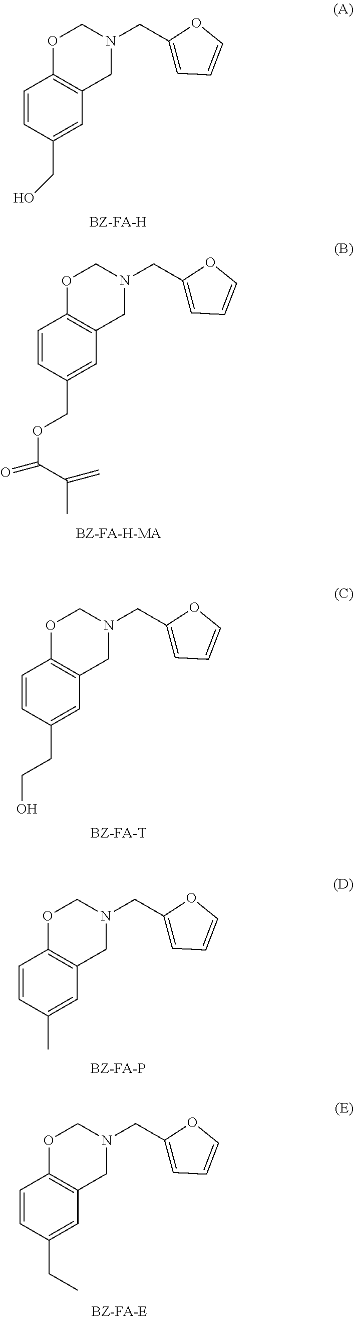 [SMILES: OCc1ccc2c(c1)CN(Cc1ccco1)CO2 ; C=C(C)C(=O)OCc1ccc2c(c1)CN(Cc1ccco1)CO2 ; OCCc1ccc2c(c1)CN(Cc1ccco1)CO2 ; Cc1ccc2c(c1)CN(Cc1ccco1)CO2 ; CCc1ccc2c(c1)CN(Cc1ccco1)CO2]

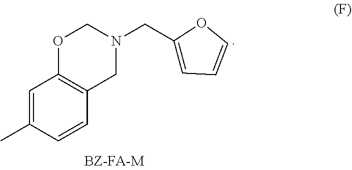 [SMILES: Cc1ccc2c(c1)OCN(Cc1ccco1)C2]

61-64. (canceled)
